(19) United States
(12) Patent Application Publication
(10) Pub. No.: US 2009/0169478 A1
(43) Pub. Date: Jul. 2, 2009

(54) In Vivo Imaging and Therapy with Magnetic Nanoparticle Conjugates

(75) Inventors: Carola Leuschner, Baton Rouge, LA (US); Challa S.S.R. Kumar, Baton Rouge, LA (US); William Hansel, Baton Rouge, LA (US); Josef Hormes, Baton Rouge, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(73) Assignee: BOARD OF SUPERVISORS OF LOUISIANA STATE UNIVERSITY, Baton Rouge, LA (US)

(21) Appl. No.: 11/997,370

(22) PCT Filed: Aug. 4, 2006
(86) PCT No.: PCT/US06/30630

§ 371 Date: Jan. 15, 2009

Related U.S. Application Data
(60) Provisional application No. 60/706,800, filed on Aug. 9, 2005.
(60) Provisional application No. 60/735,523, filed on Nov. 10, 2005.

(51) Int. Cl. A61K 49/18; B32B 1/00; A61K 49/04; A61K 49/22; A61K 9/14; A61K 39/395; A61P 35/00

(52) U.S. Cl. 424/9.3; 428/402; 424/9.4; 424/9.5; 424/489; 424/130.1; 977/773

(57) ABSTRACT

A non-invasive in vivo technique is disclosed, useful for example in detecting cancers and micrometastases. The technique may be used to selectively deliver drugs to target cells such as tumors, metastases, micrometastases, and individual malignant cells. Ligands with specificity for a target cell receptor, and optionally drug molecules as well, are covalently bound to magnetic nanoparticles, either directly or through a spacer molecule. The ligand precludes the need for a separate coating layer. For example, human breast cancer cells express receptors both for luteinizing hormone/chorionic gonadotropin (LH/CG), and for luteinizing hormone releasing hormone (LHRH). These cells can be specifically targeted by iron oxide nanoparticles covalently linked to LH/CG or LHRH. The nanoparticles are incorporated into the cancer cells through receptor-mediated endocytosis. The specific accumulation in targeted cancer cells enhances resolution for imaging, therapy, or both. The ligand may, for example, be a hormone, receptor, or antibody, or a fragment thereof.

(In countries other than the United States:) The benefit of the 9 Aug. 2005 filing date of U.S. provisional patent application Ser. No. 60/706,800, and of the 10 Nov. 2005 filing date of U.S. provisional patent application Ser. No. 60/735,523 are claimed under applicable treaties and conventions. (In the United States:) The benefit of the 9 Aug. 2005 filing date of U.S. provisional patent application Ser. No. 60/706,800, and of the 10 Nov. 2005 filing date of U.S. provisional patent application Ser. No. 60/735,523 are claimed under 35 U.S.C. § 119(e). In addition, the present application is also a continuation-in-part under 35 U.S.C. § 120 of U.S. nonprovisional patent application Ser. No. 10/816,732, filed 2 Apr. 2004. (In all countries) The entire disclosures of each of these three prior applications are hereby incorporated by reference.

The development of this invention was partially funded by the United States Government under grants R01EB002044 and R01GM61915 awarded by the National Institutes of Health, and under grant number NSF/LEQSF (2001-04) RII-03 awarded by the National Science Foundation. The United States Government has certain rights in this invention.

This invention pertains to the target-specific delivery of nanoparticles to tissues and cells, and their accumulation in targeted tissues and cells for imaging, and for therapy. The invention is useful, for example, in high-resolution, non-invasive in vivo imaging of tumors, metastases, cardiovascular system, angiogenesis, and diseased joints. The invention is also useful, for example, in selectively destroying cells in tumors and metastases, or other selected cells such as neovasculature or inflammatory cells.

Mammary adenocarcinoma is the second leading cause of cancer deaths in women. At the time of diagnosis 20-40% of breast cancer patients already have occult metastases. Bone and lymph node metastases have already occurred in 26% of mammary adenocarcinomas at the time of initial diagnosis. Removal of the primary tumor can promote metastatic growth. Bone is the most common site of metastasis for breast cancers. It has been reported that following removal of the primary tumor, up to 80% of patients develop metastatic disease in the bones. More than 70% of breast cancer deaths result from skeletal metastases. The presence of lymph node metastases is not correlated with the presence of metastases in bones or lung. Hence, the absence of lymph node metastases is a poor predictor for bone marrow or peripheral organ metastases.

There is an unfilled need for sensitive methods to image tissues in vivo non-invasively. As one example, there is an unfilled need for methods to detect tumors, disseminated cancer cells and micrometastases. Early diagnosis followed by early intervention allows steps to be taken to help to delay the progress and differentiation of metastases into overt tumors. There are currently no reliable methods for in vivo imaging of tumors smaller than about 1 cm in diameter. The accurate diagnosis of metastatic disease can be crucial to the outcome for cancer patients. There is also an unfilled need for improved methods to selectively kill cells in tumors and metastases.

One method to diagnose cancers is magnetic resonance imaging (MRI). The resolution of existing MRI techniques is limited for detecting micrometastases in peripheral tissue, or to image small primary tumors. The sensitivity of MRI can be increased with contrast agents such as paramagnetic gadolinium oxide particles, superparamagnetic iron oxide nanoparticles (SPIONs), or other magnetic nanoparticles.

SPIONs are taken up by macrophages and are delivered by the reticulo endothelial system into healthy cells. Contrast for imaging results from the higher concentration of nanoparticles in healthy cells than in malignant cells; but this system is not well-suited for imaging small areas of malignancy outside the reticulo endothelial system. Injected nanoparticles tend to have a short circulation time in vivo, and accumulate in organs of the reticulo endothelial system, including liver, spleen, kidneys, and bone marrow. Because iron oxide nanoparticles are rapidly opsonized in vivo, coatings such as dextran have been used to help inhibit opsonization, which helps to extend circulation times somewhat.

Previous methods for detecting tumor metastases in vivo have lacked sufficient sensitivity to detect micrometastases or single disseminated cells and cell clusters. Such prior methods have included, for example, counting macroscopic nodules or tumor cell colonies in histological sections, a method that has a detection rate of only about 1-2%. Immunocytochemistry techniques have detected about 30% of metastases in bone marrow aspirates. RT-PCR (Reverse transcriptase-polymerase chain reaction) techniques have been reported to detect cytokeratin 18 in a single cell mixed in 2×10⁷ bone marrow cells. These techniques are superior to histological examinations, but they still rely on highly invasive procedures such as bone marrow aspiration. Additionally, it can take up to seven days to characterize biopsy samples.

Non-invasive imaging techniques besides MRI include positron emission tomography (PET), computer tomography (CT), magnetic spectroscopy, X-ray, and ultrasonic imaging. MRI and CT techniques are not dependent on tissue depth, and do not require radioisotopes.

Gadolinium and magnetite nanoparticles have been used as contrast-enhancing agents for magnetic resonance imaging. Depending on the properties of the contrast agents, the T1 (longitudinal) or T2 (transverse) weighted images or both may be altered. Methods to increase the resolution of MRI imaging include: extending the scan time, using high efficiency coils, increasing field strength, and increasing the accumulation of contrast agent in cells or tissue.

MRI contrast agents have been tested in imaging of the liver, spleen, gastrointestinal tract and their cancers, detection of other cancers, and cardiovascular disease. When administered systemically, nanoparticles typically accumulate in the liver, spleen, and bone marrow, all of which are dependent on the reticulo endothelial system (RES). Furthermore, prior contrast agents have generally labeled healthy cells rather than malignant cells, making it difficult to identify small tumors and metastases. This “filtering” of nanoparticles has generally limited their use for imaging to the specific tissues in which they accumulate. For example, Endorem™ and AMI25™, dextran-coated iron oxide particles ˜62-150 nm diameter, have been used clinically for liver diagnostics; up to 80% of these particles accumulate in the liver. The circulation half-life can be increased by using particles smaller than 50 nm. AMI25™ iron particles have also been tested for tumor imaging in bone marrow.

Unmodified iron oxide nanoparticles that are injected into biological systems are rapidly coated with plasma proteins (“opsonization”), and then form aggregates. Opsonized particles are quickly recognized by the macrophages and mononuclear phagocytic system of the RES (reticulo endothelial system), which transport them to the liver, spleen, lymph nodes, nervous system (microglia), and bones. The nanoparticles are typically cleared from the circulation within minutes, preventing access to peripheral tissue or tumor tissue, and limiting the particles' use as contrast agents in tissues other than those in which they accumulate.

Various coatings and reduced particle size (below ˜100 nm) have been used to mask the nanoparticles from the mononuclear phagocytic system, thereby increasing their circulation time and access to tumors. The nanoparticles can preferentially accumulate in tumors because of their hyperpermeable vasculature. However, cellular accumulation has previously been lower than would be desirable, and there remains an unfilled need for improved ways to enhance the cellular uptake of magnetic nanoparticles.

Prior workers have, for example, coated iron oxide particles with a layer such as dextran to inhibit opsonization, and have then attached cell-specific ligands to the coating. For example, it has been reported that the uptake of 45 nm iron oxide nanoparticles by lymphocytes was substantially improved by coating the iron oxide particles with dextran, and attaching the HIV tat peptide ligand to the dextran coating. See C. Dodd et al., “Normal T-cell response and in vivo magnetic resonance imaging of T cells loaded with HIV transactivator-peptide-derived superparamagnetic nanoparticles,” J. Immunol. Meth., vol. 256, pp. 89-105 (2001).

Antibodies and other ligands have been attached to coated magnetic nanoparticles. See, e.g., Winter et al., “Molecular imaging of angiogenesis in nascent Vx-2 rabbit tumors using novel αᵥβ₃-targeted nanoparticles and 1.5 Tesla magnetic resonance imaging,” Cancer Res., vol. 63, pp. 5838-5843 (2003). However, there has been only limited improvement in resolution with these contrast agents. To the knowledge of the inventors, no previous method has successfully resulted in substantial intracellular accumulation of the contrast agent particles, particularly in malignant cells.

It has been reported that MCF-7 cancer cells could be targeted in vitro by the cellular incorporation of magnetic nanoparticles. E. Bergey et al., “DC magnetic field induced magnetocytosis of cancer cells targeted by LH-RH magnetic nanoparticles in vitro,” Biomedical Microdevices, vol. 4, pp. 293-299 (2002).

There has also been a recent report of particles that specifically accumulate in the lymph nodes. In clinical trials occult lymph node metastases as small as 2-6 mm from prostate cancer patients have been diagnosed using dextran-coated nanoparticles. The tumor cells were not labeled, and detection was therefore indirect. M. Harisinghani et al., “Noninvasive detection of clinically occult lymph node metastases in prostate cancer,” New Engl. J. Med., vol. 348, pp. 2491-2499 (2003).

Some previous contrast agents have permitted site specificity, and have accumulated either on the tumor surface or to a limited extent within the tumor cells. But to the knowledge of the inventors, no prior contrast agents have permitted both site specificity, and internalization of large amounts of a contrast agent within cells, i.e., amounts sufficient to substantially enhance imaging. There would be enormous benefit if contrast agents could be delivered to specifically targeted cells, and if the cells would internalize the contrast agents in sufficient amounts to substantially enhance imaging of the targeted cells in vivo.

T. Suwa et al., “Magnetic resonance imaging of esophageal squamous cell carcinoma using magnetite particles coated with anti-epidermal growth factor receptor antibody,” Int. J. Cancer, vol. 75, pp. 626-634 (1998) noted that “magnetite has the capacity to adsorb or attach chemically to inert material without changing the characteristics of T2 relaxivity . . . ” The authors disclosed coating super-paramagnetic magnetite nanoparticles with BSA and monoclonal antibodies directed against EGF receptors, which are over-expressed in esophageal squamous cell carcinoma. MRI data indicated that the nanoparticles were present in the tumor cells, and electron microscopy showed that the nanoparticles were in the lysosomes. However, MRI imaging of tumors was not substantially improved by the nanoparticles; the authors concluded that the density of target receptors was too low for sufficient uptake of magnetic nanoparticles to improve MRI imaging of tumors.

H. Choi et al., “Iron oxide nanoparticles as magnetic resonance contrast agent for tumor imaging via folate receptor-targeted delivery,” Acad. Radiol., vol. 11, pp. 996-1004 (2004) discloses the production of dextran-coated iron oxide particles, with the dextran coating covalently linked to N-hydroxysuccinimide-folate and fluorescent isothiocyanate. Endocytosis into targeted carcinoma cell lines expressing folate receptors was rapid with the folate-conjugated particles, but not with control particles lacking the folate. Endocytosis was not seen in a different carcinoma cell line lacking folate receptors. In vivo imaging of a 1 cm tumor in a mouse was reported.

Other reported examples include magnetic nanoparticles conjugated to LHRH with a silica coating in MCF-7 cells, and transferrin-conjugated monocrystalline iron oxide particles with a dextran coating. However, transferrin-receptor targeting did not appear to result in receptor-mediated endocytosis of the particles. Antibody-coupled paramagnetic liposomes targeting integrin αᵥβ₃ of endothelial vascular cells were reported to increase MRI imaging of angiogenic vasculature in rabbit carcinoma. See Winter et al., (2003) and Bergey et al. (2002).

J. Bulte, “In vivo tracking of magnetically labeled cells,” J. Cereb. Blood Flow Metab., vol. 22, pp. 899-907 (2003) reported that magnetic resonance imaging with magnetic nanoparticles can achieve a resolution of 20-25 micron, approaching the size of a single cell.

J. Kukowska-Latallo et al., “Nanoparticle targeting of anti-cancer drug improves therapeutic response in animal model of human epithelial cancer,” Cancer Res., vol. 65, pp. 5317-24 (2005), reported that a nanoparticle (dendrimer)-targeted drug concentrated in cancer cells in a kidney cancer model, with biological effects on the tumors. Methotrexate, fluorescent agents, or folic acid were attached to dendrimers of polyamidoamine. A significant reduction in tumor growth was reported as compared with methotrexate or dendrimer alone.

Y. Tsang et al., “Hepatic micrometastases in the rat: ferrite-enhanced MR imaging,” Radiology, vol. 167, pp. 21-24 (1988) discloses the use of ferrite-enhanced magnetic resonance imaging to detect hepatic metastases smaller than 1 cm in rats.

Y. Zhang, “Surface modification of superparamagnetic magnetite nanoparticles and their intracellular uptake,” Biomaterials, vol. 23, pp. 1553-1561 (2002) discloses that receptor-mediated endocytosis can cause ligand-modified magnetic nanoparticles to be taken into cells.

M. Bellin et al., “Iron oxide-enhanced MR lymphography: initial experience,” Eur. J. Rad., vol. 34, pp. 257-264 (2000) discloses the use of ultrasmall superparamagnetic iron oxide particles as contrast agents to enhance imaging in intravenous magnetic resonance lymphography. Iron oxide crystals 4.3-6.0 nm were coated with a low-molecular weight dextran to inhibit uncontrolled aggregation of the magnetic crystals.

G. Lanza et al., “Targeted antiproliferative drug delivery to vascular smooth muscle cells with a magnetic resonance imaging nanoparticle contrast agent: Implications for rational therapy of restenosis,” Circulation, pp. 2842-2847 (2002) discloses the use of antibody-targeted paramagnetic nanoparticles to deliver doxorubicin or paclitaxel to vascular smooth muscle cells, and to enhance magnetic resonance imaging.

S. Flacke, “Novel MRI contrast agent for molecular imaging of fibrin,” Circulation, pp. 1280-1285 (2001) discloses the use of anti-fibrin antibodies in gadolinium-containing paramagnetic nanoparticles to enhance magnetic resonance imaging of thrombus within fissures of atherosclerotic plaques.

D. Sipkins et al., “Detection of tumor angiogenesis in vivo by αᵥβ₃-targeted magnetic resonance imaging,” Nature Med., vol. 4, pp. 623-626 (1998) discloses the use of an antibody to the angiogenesis marker endothelial integrin αᵥβ₃ to enhance magnetic resonance imaging with a paramagnetic contrast agent. See also S. Anderson et al., “Magnetic resonance contrast enhancement of neovasculature with αᵥβ₃-targeted nanoparticles,” Mag. Res. in Med., vol. 44, pp. 433-439 (2000).

D. Chen et al., “Preparation and characterization of YADH-bound magnetic nanoparticles,” J. Mol. Cat. B: Enzym., vol. 16, pp. 283-291 (2002) discloses the binding of yeast alcohol dehydrogenase (YADH) to iron oxide particles (mean diameter 10.6 nm). The motivation was to improve the stability of the YADH enzyme. The conjugates were prepared by co-precipitating Fe⁺² and Fe⁺³ in ammonia solution, and treating under hydrothermal conditions, followed by carbodiimide activation. Binding of YADH to the nanoparticles was confirmed with Fourier transform infrared spectroscopy. It was suggested that —NH₂ ligands might be present on the surface of the Fe₃O₄ nanoparticles. The authors noted that a characteristic band of —NH₂ at 1625 cm⁻¹ was observed in naked Fe₃O₄ nanoparticles, but not after YADH binding, suggesting that there had been a reaction between the amine group on Fe₃O₄ nanoparticles and the carboxyl group of YADH after carbodiimide activation. The authors reported that the bound enzyme retained most of its residual activity, and had better storage and thermal stability than the free enzyme.

Published U.S. patent application 2003/0082237 discloses a method for preparing nanoparticles, including magnetic nanoparticles, that were said to be useful, for example, for drug delivery or for imaging.

Published international patent application WO 03/022360 (2003) discloses the use of magnetic particles attached to target-specific ligands to destroy target cells by hyperthermia. Methods of imaging nanoparticles within the body are also mentioned. Limited in vitro experimental data were presented.

C. Kumar et al., “Magnetic nanoparticle bound lytic peptide conjugates,” presentation at Louisiana Materials Research and Development Conference (Lafayette, La., Nov. 5, 2003) discloses the therapeutic use of ligand-lytic peptide magnetic nanoparticle conjugates against cancer cells.

C. Kumar et al., “Efficacy of lytic peptide-bound magnetic nanoparticles in destroying breast cancer cells,” J. Nanosci. Nanotech., vol. 4, pp. 245-249 (April 2004) discloses the characterization of hecate-conjugated magnetite nanoparticles, and their therapeutic use against breast cancer cells in vitro.

F. Sagnang, “Nano-wars. Targeting cancer cells with nano-particles,” Innovation (December 2004) describes certain work and images that arose from the present invention. See also J. Zhou et al., “Functionalized magnetic nanoparticles for early breast cancer detection,” Abstract, TMS Annual Meeting (2005).

K. Shannon et al., “Simultaneous acquisition of multiple orders of intermolecular multiple-quantum coherence images in vivo,” Magnetic Resonance Imaging, vol. 22, pp. 1407-1412 (2004) discloses a technique for NMR imaging with multiple intermolecular multiple-quantum coherences. The authors describe demonstration experiments using earthworms, grapes, and human breast tumors in mice using LHRH-conjugated nanoparticles to label malignant tissue. The authors gave no description of the synthesis of the nanoparticles.

Ligand-lytic peptide conjugates, and their uses in applications such as destroying cancer cells, are disclosed in C. Leuschner et al., “Membrane disrupting peptide conjugates destroy hormone dependent and independent breast cancer cells in vitro and in vivo,” Breast Cancer Research and Treatment, vol. 78, pp. 17-27 (2003); and published international patent application WO 98/42365.

We have discovered a novel approach to enhance non-invasive imaging in vivo, for example detection of tumors, metastases, and micrometastases. We have also discovered a novel approach to selectively deliver drugs to cells, for example to kill cells in tumors, metastases and micrometastases. The novel imaging technique uses magnetic nanoparticles that are directly bound covalently to a ligand with specificity for a receptor on the surface of the target cells. In the novel treatment technique, in addition to the ligand one or more toxin molecules (or drug molecules) are directly bound covalently to the same magnetic nanoparticles. We have discovered that the ligand itself precludes the need for a layer to inhibit opsonization, and have discovered a method for the direct attachment of ligand to magnetic nanoparticle. For example, human breast cancer cells express receptors both for luteinizing hormone/chorionic gonadotropin (LH/CG), and for luteinizing hormone releasing hormone (LHRH). These cells can be specifically targeted by SPIONS covalently linked to LH/CG or LHRH. The nanoparticles are incorporated into cancer cells through receptor-mediated endocytosis and then accumulate in the cells, particularly in the nuclei. The specific accumulation in targeted cancer cells enhances resolution for magnetic resonance imaging (MRI) detection of metastases and disseminated cancer cells in lymph nodes and peripheral organs and tissues. In addition to MRI, the novel particles may also be used in other imaging techniques, such as X-ray imaging or CT scans. The optional toxin or drug moiety selectively kills the cells with receptors for the ligand, e.g., tumors, metastases, and micrometastases. Both imaging and therapy with the ligand-SPION-toxin/drug conjugates may be conducted simultaneously.

Surprisingly, we have discovered that these magnetic nanoparticle contrast agents do not require a coating to inhibit opsonization, other than the “coating” of the targeting agent or ligand itself (or the combination of ligand and toxin or drug). The magnetic nanoparticle preferably comprises primarily Fe₃O₄, rather than the Fe₂O₃ that has been used for most prior magnetic nanoparticles. The Fe₃O₄ particle surface contains amine groups that initially prevent agglomeration, eliminating the need for a coating such as a dextran or other intermediate coatings such as have been used in most prior magnetic nanoparticles. The Fe₃O₄ nanoparticles are positively charged (˜+28 mV), which inhibits agglomeration, whereas the LHRH−SPIONS are almost neutral. The only “coating” that need be used is the targeting agent itself, one with specificity for the target cells (or a combination of targeting agent and toxin or drug). The targeting agent, and the optional toxin or drug are covalently linked to the nanoparticle via amide linkages formed by reaction with the amine groups on the particles. The targeting agent may, for example, be a hormone, ligand, or antibody, or a fragment thereof to assist in selectively directing the particles to the cells of interest and facilitating their intracellular up-take and accumulation. The optional toxin or drug may, for example, be a lytic peptide, other peptide toxin, or other toxin or drug. Our studies have shown that reducing the amount of ligand/coating on the nanoparticles increases macrophage recognition and incorporation. By optionally distancing the ligand from the nanoparticle surface through a spacer molecule, ligand specificity may be retained while increasing cellular uptake.

Alternatively, the nanoparticle may comprise Fe₂O₃ or FeO. In lieu of the amine groups leading to amide linkages with an Fe₃O₄ nanoparticle, with an Fe₂O₃ nanoparticle hydroxyl groups may be used for ester linkages between the iron oxide nanoparticle and the covalently-bound ligand. Nanoparticles comprising Fe₂O₃/Fe₃O₄ may optionally also include a “spacer” molecule between the amine groups on the particle surface and the ligand. Such spacer molecules include, for example, dicarboxylic acids such as glutaric acid or succinic acid. The introduction of spacer molecules can improve ligand-receptor interaction, resulting in increased cellular uptake, and may also increase the stability of the attached ligand. See C. Kumar et al., “Glutaric acid as a spacer facilitates improved intracellular uptake of LHRH−SPION into human breast cancer cells,” International Journal of Nanomedicine, (2006, in review); and C. Leuschner et al., “Engineering of Iron Oxide Nanoparticles for Treatment and Improved Intracellular Accumulation in Breast Cancer Cells,” 6th International Conference on Scientific and Clinical Applications of Magnetic Carriers (May 2006, Krems, Austria).

In vivo, a relatively low fraction of the novel nanoparticles accumulate in the liver (that is, unless a liver-specific targeting agent is used). Also, they are phagocytosed at a relatively low rate. Receptor-specific endocytosis by the targeted cells is substantially higher than has been reported for prior magnetic nanoparticles. Endocytosis rates of ˜450 pg/cell have been seen in prototype experiments. Early detection of small tumors can greatly enhance a patient's survival and treatment. This method of detecting early tumors and disseminated tumor cells is independent of tumor vascularization.

For example, to image or kill a tumor whose cells express receptors for luteinizing hormone releasing hormone (LHRH, also known as gonadotropin releasing hormone, or GnRH), for example many breast cancers, ovarian cancers, prostate cancers, lung cancers, pancreatic cancers, melanomas, endometrial cancers, hepatic cancers, brain cancers, oral cancers, and non-Hodgkin's lymphoma, the nanoparticles might incorporate LHRH ligands. These nanoparticles specifically bind to tumors expressing LHRH receptors on their cell surfaces. The ligand-conjugated nanoparticles can enter the cells through receptor-mediated endocytosis rather than a non-specific process such as phagocytosis or pinocytosis. This uptake is dependent on the presence of appropriate receptors on the cell surface. Cells without the appropriate receptors only take up a smaller concentration of particles through non-specific phagocytosis.

Preferably, the magnetic nanoparticles are sufficiently small (smaller than 500 nm, preferably smaller than about 400 nm, more preferably smaller than about 200 nm, and most preferably smaller than about 100-150 nm in diameter) that they do not trigger an immune response or thrombosis. A small size also helps to enhance the half life of the particles in circulation. The size of the magnetic nanoparticles may be controlled, for example, by selection of reaction conditions such as temperature, presence and type of stabilizing agent, ratio of metallic salts to surfactants, and the like. See C. Murray et al., “Colloidal synthesis of nanocrystals and nanocrystal superlattices,” IBM J. Res. Dev., vol. 45, pp. 47-56 (2001). In prototype experiments, we have used nanoparticles ˜10 nm in diameter, and particles ˜5 nm or even ˜1 nm could be used.

For some applications, however, the particles may be as large as about 500 nm; 500 nm particles will preferentially accumulate in the liver, for example, if imaging of the liver or targeting of liver cells is desired. The particles can be manufactured to be chemically and magnetically stable, and to have a high magnetic moment. Stability may optionally be enhanced, for example, by coating the magnetic nanoparticles with a noble metal surface, although this is not preferred for most applications, due to potential toxic effects and possible interference with covalent binding to the ligand. Such a surface can improve both oxidative and magnetic stability. Methods of coating magnetic nanoparticles with a noble metal shell are known in the art. See, e.g., J. Park et al., “Synthesis of ‘solid solution’ and ‘core-shell’ type cobalt-platinum magnetic nanoparticles via transmetalation reactions,” J. Am. Chem. Soc., vol. 123, pp. 5743-5746 (2001). Stability may also be enhanced with organic stabilizers. See, e.g., H. Bönnemann et al., “A size-selective synthesis of air stable colloidal magnetic cobalt nanoparticles,” Inorganica Chimica Acta, vol. 350, pp. 617-624 (2003). The organic stabilizers inhibit agglomeration (i.e., “steric stabilization”). It is rare that organic stabilizers inhibit oxidation of a metal core, however. In the present invention, there is no need for organic stabilizers on the magnetic nanoparticles themselves, which are bound directly to the ligand and to the optional toxin or drug (electrostatic stabilization). The ligands can act both as targeting mechanisms and as coatings at the same time. Recent studies have shown that reduced ligand numbers on the nanoparticles increased macrophage uptake, and therefore RES susceptibility. It is possible that the optional toxins or drugs may act as coatings as well. The ligands can be less likely to provoke an immune response than other coatings, such as dextrans. Small peptide toxins, such as lytic peptides, can also be selected with low immunogenicity.

Superparamagnetic particles have no remnant magnetization when an applied magnetic field is removed, meaning that the particles are less likely to aggregate. Dipolar interactions between superparamagnetic nanoparticles and surrounding tissue protons help increase both T1 and T2 relaxation rates.

Although the magnetic nanoparticle comprises primarily Fe₃O₄, we do not exclude the possibility that some amount of Fe₂O₃ may be present, depending primarily on the amount of oxygen present during synthesis of the particles. It is preferred that the amount of Fe₃O₄ be at least about 50% of the total iron oxide present. Less preferred the magnetic nanoparticles may comprise more than 50% Fe₂O₃, even up to 100% Fe₂O₃.

Iron oxide nanoparticles are biologically safe. Iron homeostasis is controlled by absorption, excretion, and storage. Iron oxide nanoparticles are metabolized into elemental iron and oxygen by hydrolytic enzymes. The iron then joins normal body stores, and is subsequently incorporated into hemoglobin. Acute toxicity has not been observed in rats or in human clinical trials. The iron is incorporated into normal metabolic pathways, including iron storage, incorporation into hemoglobin, and excretion. The iron is excreted over a period of about four weeks, and does not accumulate in tissues as heavy metals can. Renal function, hepatic function, serum electrolytes, and lactate dehydrogenase all remain essentially unchanged following treatment with iron oxide nanoparticles. Serum iron levels are elevated for about 48 hours, with no significant adverse symptoms. In rats 250 mg/kg iron particles injected intravenously have been reported to cause no adverse effects, and in mice 350 mg/kg have been reported to be well tolerated. Iron oxide nanoparticles have a greater margin of safety than gadolinium particles; the ratio between an effective dose (i.e., the smallest dose at which an image could readily be taken) for iron oxide nanoparticles and LD₅₀ has been reported to be about 1:2400, while for gadolinium the ratio is closer to 1:50.

Unlike antibody-coated particles, which may be recognized by the immune system; or particles lacking targeting agents, which may potentially be endocytosed nonspecifically by a variety of cell types; nanoparticles covalently linked to appropriate ligands may be targeted with a high degree of specificity to just those cells bearing receptors for the ligand.

Magnetic nanoparticles in accordance with the present invention may be administered, for example, by injection, as their size readily allows them to pass through capillaries and into tissue.

In addition to their uses for in vivo imaging and therapy, the novel nanoparticles may also be used in vitro or ex vivo, for example in diagnosing biopsied tissues. For example, ex vivo assays can be used to identify particular receptors on fresh tumor tissues (e.g., in fresh biopsy samples) that result in substantial endocytosis; such knowledge can help to select an individualized treatment for a patient, to enhance the likelihood of a successful outcome.

In addition to using a targeting agent (e.g., hormone, ligand, receptor, antibody, or fragment thereof) on the surface of nanoparticles to target the selected cells, the particles may also optionally be guided to the target with an external magnetic field. A magnetic field can help to concentrate the magnetic nanoparticles in a region of interest, i.e., the site of a tumor. On the other hand, it can also be helpful to allow the targeting agents to locate cells having the targeted receptor, regardless of where in the body they may be, to better locate metastases that might otherwise be overlooked or be in unexpected locations. Thus, if the goal is to image a particular tumor in a particular location, it can be useful to use an external magnetic field to guide the nanoparticles to the region. Using a magnetic field to guide the nanoparticles can be difficult, and may not be advantageous in all circumstances even where technically feasible. For example, a tumor may metastasize to form additional tumors in remote areas of the body. Or the locations of small tumors in the body may otherwise not be known. If the nanoparticles are guided solely by an applied magnetic field to reach the location of the known tumor, then metastasized or other small tumors may be missed. By allowing the destination of the nanoparticles to be guided by circulation of the target-specific agent rather than by an applied magnetic field, such unknown locations of diseased tissue may also be targeted.

The present invention provides a method for the improved imaging of targeted tissue, including for example a diseased tissue such as a cancerous tissue. It may also be used to image small tumors, small tumor cell aggregates, e.g., those smaller than about 1 cm that are difficult to image noninvasively through existing technologies, or even single malignant cells. Imaging and therapy may optionally be conducted simultaneously.

Results from prototype experiments showed that it is possible to make SPION particles that were nearly monodisperse in size (˜10 nm), that are homogeneous in solution, and that did not agglomerate. They retained their superparamagnetic properties after being conjugated to ligands. LHRH−SPIONs had an almost neutral charge, while SPIONs without ligand were positively charged. We found that LHRH-conjugated SPIONs, for example, specifically targeted cells that expressed LHRH receptors. Cells lacking LHRH receptors did not bind and accumulate LHRH−SPIONs. In a mouse model of human breast cancer, primary tumors and metastases specifically accumulated up to 60% of injected LHRH−SPIONs; compared to about 8% for unconjugated SPION particles in primary tumors, and essentially no unconjugated SPIONs in metastatic lesions. The accumulation of iron in a tissue depended on the number of metastatic cells in the organ. Blocking the receptors on tumor tissues was found to inhibit the accumulation of the LHRH−SPIONs in the tissues. These observations confirmed the specificity of the LHRH−SPIONs for target tissues with LHRH receptors.

Nanoparticles have several advantages over micrometer and millimeter-sized particles for targeting tumors and malignant cells. Nanoparticles are less easily recognized by the reticulo endothelial system; they may cross tumor interstitia through pores having a cutoff size of about 400 nm; nanoparticles will typically have a longer circulation half-life. By virtue of the surface-bound ligands and the size of the nanoparticles, nanoparticles in accordance with the present invention are taken up by tumor cells through receptor-mediated endocytosis, leading to an accumulation of particles within tumor cells. Larger particles of millimeter or even micrometer size do not share these properties.

Note that in some cases different portions of a naturally-occurring hormone or other ligand may be responsible for receptor binding on the one hand, and for promoting endocytosis on the other hand. Where appropriate, it is therefore preferred to include both the portion of a naturally-occurring ligand that promotes receptor binding, as well as the portion that promotes endocytosis. For example, a 15-amino acid segment of the β-subunit of chorionic gonadotropin (CG) promotes receptor binding, but only limited endocytosis, while the full CG molecule both binds to the receptor and is efficiently endocytosed. Thus the entire CG molecule may be preferred for use as a ligand in this invention, rather than the β-subunit or a fragment of the β-subunit. Also, by including the full ligand, fewer total nanoparticles may be needed in particular applications, as they are less likely to be taken up by non-target cells.

In use for cancer therapy, a (Ligand)ₓ-Nanoparticle-(Drug)_{y} construct in accordance with the present invention has the following advantages, characteristics, optional characteristics, or preferred characteristics:
1. Specific targeting of tumors and metastases.
2. Targeted delivery of drugs to tumors, metastases, nonvascularized malignant cell clusters, and individual malignant cells.
3. Facilitation of drug uptake in target tissue.
4. Incorporation of the entire construct into targeted tumor and metastatic cells through ligand-receptor mediated endocytosis.
5. Increased accumulation of particles within the target tumor and metastatic cells.
6. Retention of nanoparticles in tumors and metastases, allowing sustained release of drug within the targeted tissues.
7. Reduced systemic side effects through specific targeting.
8. Simultaneous targeting and imaging of widespread tumors and metastases.
9. Ability to simultaneously treat and image diseased tissue with non-invasive methods, e.g., magnetic resonance imaging, positron emission tomography, ultrasound, computed tomography, single photon emission tomography.

### BRIEF DESCRIPTION OF THE DRAWINGS

FIG. 1 depicts a schematic representation of one embodiment of a ligand-coated magnetic nanoparticle in accordance with the present invention, in which LHRH is the ligand.

FIG. 2 presents a schematic depiction of the binding process, using LHRH as an example ligand that is bound to an iron oxide nanoparticle.

FIGS. 3(a) through (d) present photographs of lung sections with metastases from breast cancer xenografts, with or without nanoparticles.

FIG. 4 depicts toxicity measurements of different constructs on three cell lines.

FIG. 5 depicts the time course of treatment for tumor-xenografted mice.

FIG. 6 depicts tumor weights at necropsy for tumor-xenografted mice, following various courses of treatment.

FIGS. 7(a) and (b) depict the changes in tumor volume, and the absolute tumor mass at necropsy, respectively, for tumor-xenografted mice, following various courses of treatment. Tumor volumes and tumor weights decreased only in mice that had been injected with LHRH−SPION−Hecate or LHRH-Hecate.

FIGS. 8(a), (b), and (c) depict body mass, liver mass, and gonadal mass at necropsy, respectively, for tumor-xenografted mice, following various courses of treatment.

FIG. 9 depicts measurements of lymph node metastases, assayed by luciferase activity, at necropsy for tumor-xenografted mice, following various courses of treatment. Lymph node metastases were destroyed after treatments with LHRH-Hecate and LHRH−SPION−Hecate.

FIG. 10 depicts iron accumulation in lymph nodes in mice following various courses of treatment. The observed iron accumulation in lymph nodes in LHRH−SPION−Hecate-treated Mice was comparable to that following LHRH−SPION injections.

FIG. 11 depicts one embodiment of the novel nanoparticles with a “spacer” molecule between the amine groups on the particle surface and the ligand.

FIG. 12 depicts the effect of introducing “spacer” molecules on endocytosis.

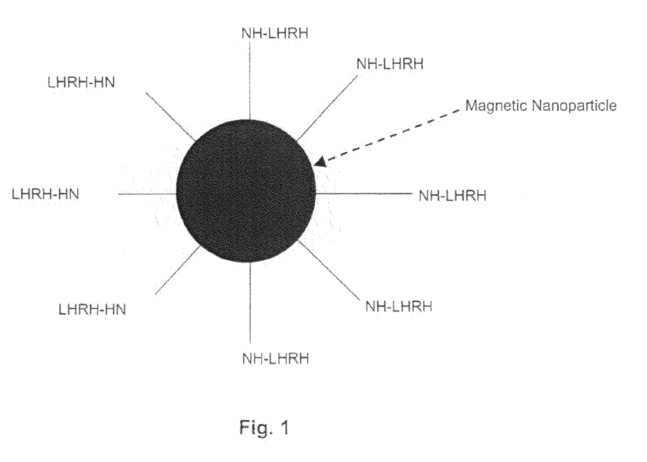

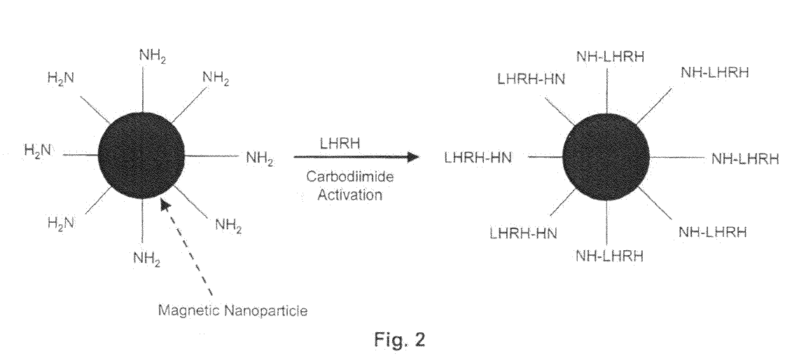

Models for Studying Metastases. In vitro models of breast cancer metastases were used in some studies. The human breast cancer cell line (MDA-MB-435S) was isolated from a human ductal carcinoma from a pleural metastatic site, and is commercially available from the American Type Culture Collection. This cell line has served as a model in other studies. MDA-MB-435S cells are estrogen-independent; they express receptors both for LH/CG and for luteinizing hormone releasing hormone (LHRH); and they are highly metastatic in nude mice. Lymph node, lung, and bone metastases have been observed in nude mice with MDA-MB-435S xenografts. The nude mouse has no T-cells, and does not reject xenografted human tumor cells. The nude mouse is commonly used as a model for several types of human cancers.

We have studied in detail the behavior of MDA-MB-435S xenografts in nude mice. Tumor cells were injected along with Matrigel. More than 90% of the xenografted mice developed tumors. Vascularization of the primary tumor was observed as early as ten days after tumor cell inoculation. Using Matrigel with subcutaneous tumor cell inoculations increased the metastatic behavior of the xenografts. The high metastatic potential of MDA-MB-435S breast cancer cells in nude mice makes it a good model for studying the effects of anti-cancer drugs in vivo. The MDA-MB-435S cell line, stably transfected with the luciferase gene, has been used as a tool in investigating micrometastases and disseminated tumor cells. Micrometastases and tumor cell clusters in peripheral organs, lymph nodes and bones can be quantified after necropsy in individual organs by measuring luciferase activity in tissue homogenates.

### EXAMPLE 1

N-ethyl-N′(3-dimethylaminopropyl)carbodiimide hydrochloride, FeCl₃, FeCl₂.4H₂O, and NH₄OH (28%, aqueous) were purchased from Aldrich. The water used throughout all Examples was or is “nanopure” water, unless otherwise stated. The “nanopure” water was produced by a Barnstead nanopure water purification system. Dissolved oxygen was removed by refluxing the water under nitrogen for three days.

Magnetite nanoparticles were synthesized under inert atmospheric conditions. FeCl₃ (1.622 g) and FeCl₂.4H₂O (0.994 g) were placed In a three-necked, 100 mL round bottom flask. To remove any traces of O₂ from the flask, the flask was then evacuated and purged with nitrogen three times. The iron salts were dissolved in 25 mL water under nitrogen, and the solution was stirred magnetically. To this solution, 2.5 mL of 28% NH₄OH was added dropwise at room temperature. A black precipitate was produced. The precipitate was heated at 80° C. for 30 minutes, washed several times with water, then washed with ethanol, and then dried in a vacuum oven at 70° C. Although it is preferred to carry out this preparation in an inert atmosphere, it may also be conducted in the presence of free oxygen, and even under normal atmospheric conditions.

### EXAMPLE 2

We have demonstrated that LHRH-conjugated magnetic nanoparticles preferentially accumulate in tumor tissue as compared to normal tissue (i.e., essentially any non-malignant tissue other than gonadal tissue; in our experiments, kidney tissue was used as the normal tissue for comparison). This experiment employed male nude mice bearing tumors from human prostate cancer line PC-3.luc (with luciferase reporter). The PC-3 cell line, available from the American Type Culture Collection, was established from a prostate-to-bone metastasis in a male patient. This cell line was transfected with the luciferase gene from the Photinus pyralis firefly by lipofection. See N. Rubio et al., “Traffic to lymph nodes of PC-3 tumor cells in nude mice visualized using the luciferase gene as a tumor cell marker,” Lab. Invest., vol. 78, pp. 1315-1325 (1998); N. Rubio et al., “Metastatic burden in nude mice organs measured using prostate tumor PC-3 cells expressing the luciferase gene as a quantifiable tumor cell marker,” Prostate, vol. 44, pp. 133-143 (2000).

### EXAMPLE 3

Fe₃O₄ nanoparticles were bound to LHRH by the following procedure. LHRH with free carboxlic acid was purchased from Bachem (www.bachem.com). Magnetite nanoparticles (60 mg) prepared as in Example 1 were dispersed in 6 ml of water by sonication under nitrogen. A freshly prepared carbodiimide solution (42 mg in 1.5 ml of water) was added, and the solution was sonicated an additional 10 minutes. The mixture was cooled to 4° C., and a solution of 3.7 mg LHRH in 1.5 ml of water was added. The reaction temperature was maintained at 4° C. for 2 hours with occasional swirling of the flask. After 2 hours, the flask was placed on a permanent magnet, and the LHRH-bound magnetic nanoparticles settled out. The supernatant was analyzed for unbound LHRH by quantitative HPLC. The LHRH-bound nanoparticles were washed three times with water, followed by washing with ethanol, and were then dried under a slow stream of nitrogen.

### EXAMPLE 4

Fe₃O₄ nanoparticles were bound simultaneously to LHRH and hecate by the following procedure. LHRH with free carboxlic acid was purchased from Bachem (www.bachem.com). Hecate with free carboxylic acid was obtained from the protein crystallographic facility at Louisiana State University (Baton Rouge, La.). Magnetite nanoparticles (60 mg) prepared as in Example 1 were dispersed in 6 ml of water by sonication under nitrogen. A freshly prepared carbodiimide solution (42 mg in 1.5 ml of water) was added, and the solution was sonicated an additional 10 minutes. The mixture was cooled to 4° C., and a solution containing 1.85 mg LHRH and 1.85 mg Hecate in 1.5 ml of water was added. The reaction temperature was maintained at 4° C. for 2 hours with occasional swirling of the flask. After 2 hours, the flask was placed on a permanent magnet, and the LHRH- and hecate-bound magnetic nanoparticles settled out. The supernatant was analyzed for unbound LHRH by quantitative HPLC. The LHRH- and hecate-bound nanoparticles were washed three times with water, followed by washing with ethanol, and were then dried under a slow stream of nitrogen.

By changing the concentrations of ligand (e.g., LHRH) and toxin or drug (e.g., hecate) employed in the carbodiimide reaction, the ratios of the moieties bound to the nanoparticles may be altered as desired.

### EXAMPLE 5

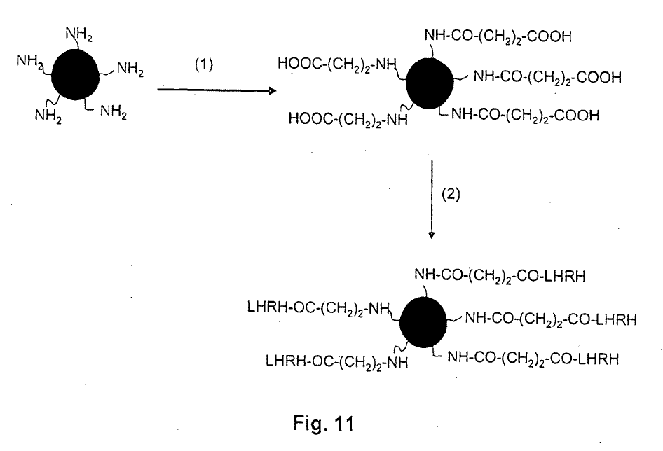

Magnetite nanoparticles with ligands and spacers were prepared as follows: Iron II chloride (FeCl₂.4H₂ 0) 98%, iron III chloride (FeCl₃) 97%, ammonium hydroxide (NH₄OH) 29.05%, 1-ethyl-3-(3-dimethylaminopropyl)carbodiimide hydrochloride (EDC), and glutaric acid were purchased from Sigma Aldrich. Air-free nanopure water was made in the lab by refluxing nanopure water, made with a Barnstead NanoPure Water System, under inert atmosphere. During the synthesis of the nanoparticles a VWR 750D Sonicator was used, as well as a VWR 1160A PolyScience Chiller. The SPIONs were prepared as otherwise described in Example 3, except as specified. For the covalent attachment of glutaric acid to the SPIONs, 60 mg of magnetite nanoparticles were dispersed in 6 ml of water with a sonication bath at room temperature for fifteen minutes. A solution of 42 mg carbodiimide and 1.5 ml water was added. The mixture was sonicated for 10 more minutes and then cooled to 4° C. in a chiller. A solution of 3.7 mg glutaric acid in 1.5 ml of water was added, and the reaction temperature was maintained at 4° C. for 2 h more. The particles were then allowed to settle on a permanent magnet. The supernatant was removed and the particles were washed three times with water, twice with ethanol, and dried under nitrogen. See FIG. 11, depicting schematically the synthesis of one embodiment of nanoparticles incorporating spacers between ligand and nanoparticle. Spacers may also be used between toxin or drug and nanoparticle. The spacer may be any moiety that covalently links, but places some distance between the nanoparticle surface and the toxin, drug, or ligand. Preferably the linker is relatively inert after bonding both to the nanoparticle and to the toxin, drug, or ligand. For example, the conjugate may take the form (nanoparticle)-NH—CO—R—CO-ligand, or (nanoparticle)-NH—CO—R—CO-toxin, or (nanoparticle)-NH—CO—R—CO-drug, or toxin-CO—R—CO—NH(nanoparticle)-NH—CO—R—CO-ligand, or drug-CO—R—CO—NH(nanoparticle)-NH—CO—R—CO-ligand; wherein R may, for example, take the form —(CHX)ₙ, where X is —H or —OH. Although it is preferred to carry out this preparation in an inert atmosphere, it may also be conducted in the presence of free oxygen, and even under normal atmospheric conditions.

### EXAMPLE 7

The procedure of Example 6 was followed to functionalize the glutaric acid-bound SPIONs with LHRH, substituting 3.7 mg of LHRH for glutaric acid, and 60 mg of glutaric acid-SPIONs instead of plain magnetite.

### EXAMPLE 8

The lateral tail vein of each MDA-MB-435S.luc xenograft-bearing mouse (6 mice per group) was injected with 250 mg/kg of a saline suspension of the LHRH-Fe₃O₄ nanoparticles (LHRH−SPION) or of Fe₃O₄ nanoparticles without LHRH (SPION). The mice were euthanized 20 hours after the injections; and their tumors, lungs, liver, heart, kidney, pancreas, spleen, and gonads were collected. Portions of the organs and tumors were embedded in paraffin and tested for iron using Prussian blue staining. Four of six tumors from mice that had been injected with the nanoparticle-bound LHRH tested positive for iron, while no tumors from the control nanoparticle-only group (without LHRH) tested positive for iron.

### EXAMPLE 9

Portions of the tissue samples from Example 8 were homogenized, and iron content was determined in calorimetric assays. The amounts of accumulated iron per gram total organ mass could thus be determined. A quantitative estimate of the iron content in tumor and kidney showed that up to 70% of the iron was located in the tumor when nanoparticle-bound LHRH was injected. When nanoparticles alone were injected, the iron content in tumors was only about 4%. The magnetic nanoparticles without LHRH were observed to accumulate in the kidney in preference to tumor, but nanoparticles with LHRH preferentially bound to the tumor, whose cells expressed LHRH receptors.

### EXAMPLE 10

We have also used LHRH- or βLH-bound magnetic nanoparticles to detect single cancer cells in in vitro assays. These results showed that the novel technique may be used in the sensitive detection of very early tumors and micrometastases. In the in vivo experiments described in Examples 8 and 11, metastatic cancer cells in the lung were individually labeled with the iron oxide nanoparticles.

LHRH−SPIONs and βCG/LH-SPIONs were tested on breast cancer cells MDA-MB-435S, on Chinese Hamster Ovary cells, on rat LH-receptor-transfected Chinese Hamster Ovary cells, and on mouse Sertoli cells. Experiments were conducted in the presence of LHRH, βCG, or neither. This in vitro study confirmed that the ligand-mediated uptake of LHRH−SPIONs and βCG/LH-SPIONs was substantially lower in cell lines that did not express the appropriate receptors. (E.g., Chinese Hamster Ovary cells do not express LH-receptors; and mouse Sertoli cells do not express LHRH receptors). In addition, we observed that co-incubation with the free peptide ligands blocked the receptors that otherwise enabled the ligand-conjugated SPIONs to enter cells through receptor-mediated endocytosis. The uptake was then primarily via phagocytosis or pinocytosis at a low rate, similar to that for unconjugated SPIONs. The observed uptake was specific; the quantity of iron nanoparticles within the cells was substantially higher for receptor-mediated uptake. For example, for the MDA-MB-435S cells the observed uptakes were 452 pg Fe/cell with LHRH−SPION, and 203 pg Fe/cell with βCG-SPION; but only 40-50 pg Fe/cell in the presence of LHRH or βCG, or when unconjugated SPIONs were used.

### EXAMPLE 11

In vivo studies are conducted in Balb/c athymic nude mice to create MDA-MB-435S.luc xenografts and metastases. Metastases may be detected in homogenates from lymph nodes, bones, and peripheral organs using the luciferase assay. MDA-MB-435S.luc xenografts are propagated subcutaneously in 48 female nude mice (6 weeks of age) from a Matrigel suspension containing about 1×10⁶ cells. The mice are monitored daily, and tumor volumes are determined by microcaliper measurements 3 times per week. Body weight is measured once a week. At a tumor volume of 50 mm³ (˜14 days post-tumor propagation) the mice are randomly allotted to 4 different treatment groups of 12 animals each.

Mice (10/group) with the MDA-MB-435S.luc xenografts are imaged by MRI prior to injection with nanoparticles. The mice are then injected intravenously with LHRH-nanoparticles (250 mg/kg), or nanoparticles without ligand (250 mg/kg), or saline. All mice are anesthetized after 24 hours, and undergo whole body magnetic resonance imaging. For example, a 0.6 T (25.1 MHz) superconducting magnetic resonance system can be used. The gradient strength should be about 2000 mT/m for a 6 mm field of view and a receiving signal of 500 kHz. A multisection spin-echo technique may be used (500 msec repetition time/32 msec echo time) for enhanced or non-enhanced scans. The highest sensitivity should be observed for the ligand/magnetite exposure. The data from the magnetic resonance imaging are compared to paraffin histological sections stained with Prussian blue for iron content, and to fresh samples of tumor sections analyzed for luciferase activity. In both T1 (longitudinal) and T2 (transverse) weighted images, nanoparticles increase signal intensity by brightening the image at sites where particles accumulate.

### EXAMPLE 12

In vivo magnetic resonance imaging of laboratory animals is conducted. Tumor-bearing mice or rats are prepared by means known in the art, such as those discussed above. Different groups of mice are injected with LHRH-nanoparticles or saline. Different groups are injected intravenously, intraarterially, or subcutaneously. Dosage is 2.5-250 mg/kg, suspension in saline. After 20-48 hours the animals are examined by MRI at 1.5-3.0 Tesla. These experiments will determine optimal dosage and route of administration, which will likely vary for different types of cancers.

### EXAMPLE 13

Groups of nude mice or rats bearing breast cancer or prostate cancer xenograft transfected with the luciferase gene are injected with ligand-bearing magnetic nanoparticles, magnetic nanoparticles without ligand, or saline. The injection may be administered intravenously, intra-arterially, or subcutaneously. After 2, 20, and 48 hours the animals are anesthetized, and nanoparticle distribution is determined by magnetic resonance imaging. The animals are then euthanized and necropsied. Individual organs, bones, and lymph nodes are examined for iron content. The number of tumor cells in the samples is determined by luciferase assay. The necropsy data are compared with the results previously obtained by MRI imaging to confirm the sensitivity of the ligand-nanoparticle imaging method.

### EXAMPLE 14

The Breast Cancer Metastasis Model We have developed an animal model for breast cancer metastases in female athymic nude mice. MDA-MB-435S.luc cells were inoculated as a suspension in Matrigel into the interscapular region. The MDA-MB-435S cells produced solid, vascularized tumors within 10 days after subcutaneous injection of 1×10⁶ cells, and were found to have high metastatic potential in the mice.

The human breast cancer cell line MDA-MB-435S was transfected by lipofection with the plasmid pRC/CMV-luc, which contains the Photinus pyralis luciferase gene and an antibiotic resistance gene under transcription control of the cytomegalovirus promoter. Stably-transfected MDA-MB-435S.luc cells were selected by exposing the cells to 400 μg/ml of the antibiotic G418. Clones with the highest expression of luciferase were selected and characterized for their LH/CG and LHRH receptor binding capacities. The LH/CG and LHRH receptor capacities were the same for the wild type and the luciferase-transfected cell lines. An in vivo model based on MDA-MB-435S.luc xenograft allowed us to investigate lymph node, peripheral organ and bone colonization to the single cell level as a function of growth, time, and cell number of the primary tumor. Micrometastases and tumor cell clusters in peripheral organs, lymph nodes and bones could be quantified in individual organs. Metastasis distributions were determined as luciferase-positive cells in homogenates from bones, lungs, and lymph nodes from mice with and without removal of the primary tumor. Metastasis distributions were assayed 35 days after tumor inoculation. Primary tumors were then surgically removed from some of the mice. Then 64 days after the original tumor inoculation, metastasis distributions were assayed both in mice in which the primary tumor had been surgically removed, and in those in which the primary tumor had not been removed. We observed that removal of the primary tumor caused a significant increase in metastatic load in bone, lymph nodes, pancreas, uterus and oviduct, liver, and kidney (data not shown).

### EXAMPLE 15

Targeting Human Breast Cancer Xenograft and their Metastases with LHRH−SPIONs in vivo. To evaluate the distribution of LHRH−SPIONs and unconjugated SPIONs in vivo, female nude mice with human breast MDA-MB-435S.luc xenograft as described above were injected intravenously with 250 mg/kg LHRH-SPIONs or with unconjugated SPIONs, 35 days after tumor inoculation. The mice were sacrificed 20 h after injection, and the organs and tumors were collected, weighed and analyzed for iron and luciferase. Portions of the organs and tumors were homogenized and their iron content determined spectrophotometrically after reaction with Prussian blue, while other portions were analyzed after fixation by transmission electron microscopy.

The design of this cancer model was such that all tumor cells expressed luciferase, meaning that the presence of luciferase was inherently synonymous with the presence of cancer cells. By contrast, the presence of iron was not inherently synonymous with the presence of cancer cells. The goal of this experiment was to test the efficacy of the novel system at specifically delivering iron to cancer cells. The observed correlation between luciferase and iron was a measure of the efficacy of the novel system in identifying tumors and metastases.

The following groups (8 mice each) were used in this set of experiments: Tumor-bearing mice receiving saline injections; tumor-bearing mice receiving unconjugated SPION injections; tumor-bearing mice receiving LHRH−SPION injections; tumor-free mice receiving saline injections; and tumor-free mice receiving LHRH−SPION injections. Results are shown in the Table below. The figures show the percentages of iron accumulating in the specified organs following injection of LHRH-SPION or SPION at a level of 2.5 mg Fe per mouse. (The figures in each row add to less than 100%; the iron that was not found in the specified tissues was not separately accounted for). In saline controls (not shown in the table), both tumor-bearing and tumor-free mice had iron content less than 0.05%. Statistical significance for the LHRH-SPION, tumor-bearing mice figures are versus the SPION injections in tumor-bearing mice for the same organ. Statistical analyses were conducted on raw data by ANOVA. We obtained the ranks of the data, and then conducted a Kruskal-Wallis test. Differences were considered significant at the P<0.05 level. Rank data and variance stabilizing transformations were included.

| | Tumor | Lung | Liver | Kidney |
|---|---|---|---|---|
| LHRH-SPION, | 59.1 | 22.2 | 5.2 | 3.3 |
| tumor- | P < 0.00006 | P < 0.03 | P < 0.01 | P < 0.3 |
| bearing mice | | | | |
| SPION, | 7.8 | 2.3 | 54 | 3.9 |
| tumor- | | | | |
| bearing mice | | | | |
| LHRH-SPION, | | 0.9 | 5.6 | 4.1 |
| tumor-free | | | | |
| mice | | | | |

In the tumor-bearing mice, 54% of unconjugated SPIONS accumulated in the liver, compared to about 8% in the tumor. By contrast, with the LHRH−SPIONs 59% of the particles accumulated in the tumor, versus about 5% in the liver. LHRH−SPIONs in tumor-free mice accumulated about 5.6% in the liver, and 4% in the kidneys. Only 0.9% of the SPIONS accumulated in the lungs of tumor-free mice, compared to 22% in the tumor-bearing mice, suggesting a high concentration of metastatic cells in the lungs. We also observed that the level of LHRH−SPIONs that accumulated in the lungs was a linear function of the metastatic load in the lungs, as measured by luciferase activity. These observations demonstrated that the metastatic cells were successfully targeted by the LHRH−SPIONs. The iron content of as few as 6 individual metastatic cells could be detected in lung homogenates.

### EXAMPLE 16

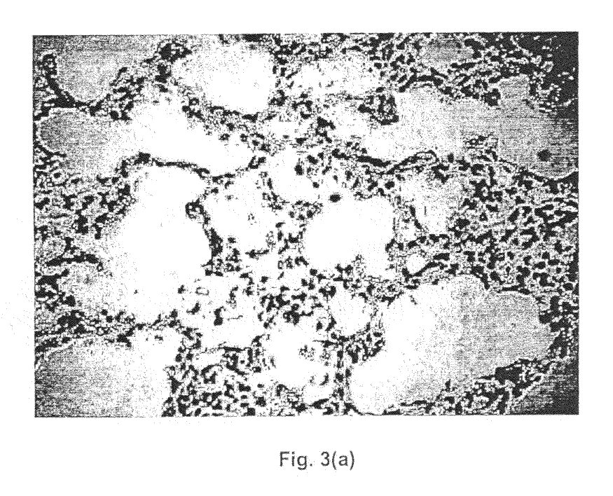

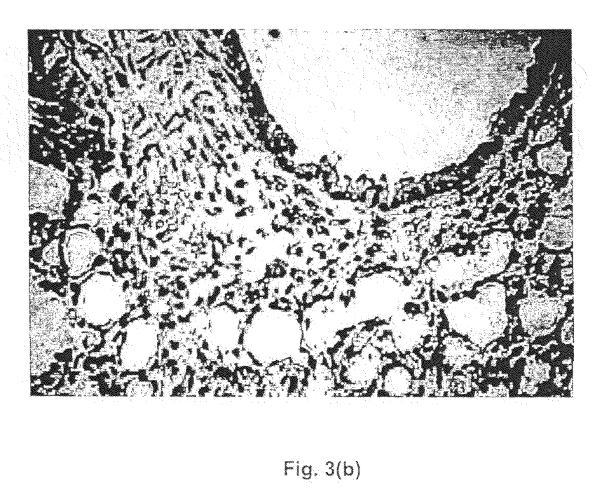

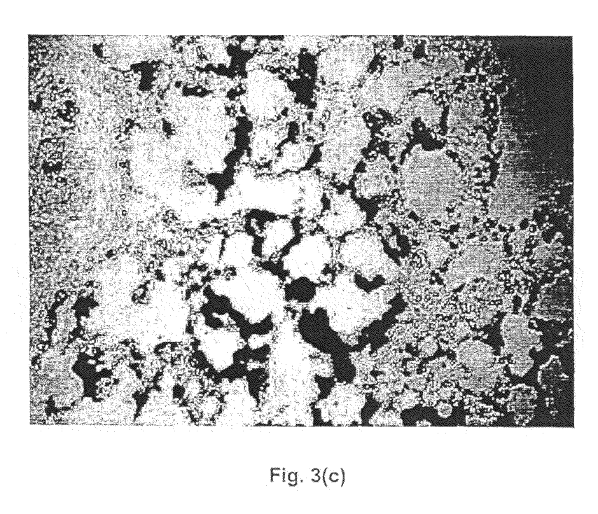

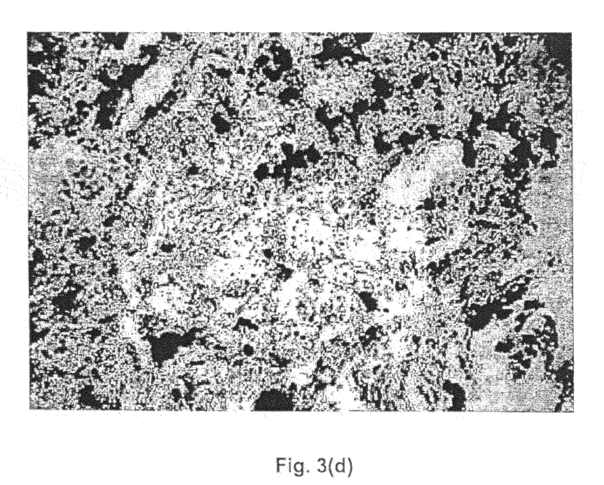

These observations were additionally confirmed by Prussian Blue staining for iron in histological slides. See FIGS. 3(a) through (d), showing photographs of Prussian Blue-stained sections from lungs of mice bearing the MDA-MB-435S.luc tumors. FIG. 3(a) depicts a saline-injected control. FIG. 3(b) depicts injection with unconjugated SPIONs. FIGS. 3 (c) and (d) depict injection with conjugated LHRH−SPIONs. Note the metastases that were clearly stained in FIGS. 3(c) and (d). The accumulated iron oxide load in tumors and metastases increased following each of three sequential injections. The nanoparticles were incorporated into the target cells and were retained inside the cells for at least four weeks.

### EXAMPLE 17

Mice with MDA-MB-435S.luc xenograft, both with and without intact primary tumors, are injected intravenously with varying concentrations of SPIONs, βCG-SPIONs, LHRH−SPIONs, and LHRH/βCG-SPIONs. The mice are then anesthetized, and magnetic resonance imaging is conducted to determine resolution limits in the early detection of breast cancer cells in vivo. Following the in vivo magnetic resonance imaging, the mice are sacrificed and accumulated magnetic nanoparticles are verified both using Prussian Blue assays from organ homogenates, and luciferase assays. Further analyses include electron energy loss spectroscopy (EELS) during TEM of the tumor and non-tumor tissues (e.g., spleen, kidney, lungs, liver, bones, lymph nodes) to determine the morphology and cellular distribution of accumulated iron particles. The actual iron content of tumors and non-tumor sites are correlated to the contrast seen in MRI images.

### EXAMPLE 18

Comparison of SPION accumulation in metastases using single- or double-ligand-conjugated SPION nanoparticles. This experiment is designed to detect metastases from MDA-MB-435S.luc xenograft with ligand-conjugated SPIONs, both in the presence and absence of the primary tumor. The MDA-MB-435S.luc cells are suspended in a Matrigel™ suspension and injected (10⁶ cells/mouse) into the interscapular region of female nude mice. This experiment uses 312 female nude mice, of which 252 receive MDA-MB-435S.luc xenograft. Primary tumors are surgically removed from some of the xenografts recipients after 25 days. A group of 60 mice without tumor inoculation serve as controls. The primary tumors are surgically removed from anesthetized mice in a sterile field under isoflurane anesthesia. The wound is closed using Michel wound clips (11 mm), which are removed 7 days post-surgery. Postoperative analgesia is also provided through standard means. The mice are housed individually in sterile cages.

Thirty-nine days after tumor cell injection (14 days after removal of the primary tumor), the mice are injected in the lateral tail vein with saline, SPIONs, βCG-SPIONs, LHRH−SPIONs, or LHRH/βCG-SPIONs (250 mg/kg per injection), with or without pre-treatment with either the same ligand, or with both ligands. After 20 hours the mice undergo MRI followed by euthanasia. Detailed necropsies are conducted. Lung, liver, kidney, spleen, tumors, ovaries, uterus, upper spine, rib cage, mid spine, lower spine, and axillary and interscapular lymph nodes are removed and weighed. The iron contents of these tissues are determined from paraffin-embedded histological sections after Prussian Blue reaction (Sigma), and quantified by spectrophotometric assays of homogenates of these organs and from fixed sections by TEM.

The metastatic load is determined through luciferase assays of organ homogenates and compared to the results from the iron determinations. Mice with saline injections and mice without tumors serve as controls and undergo the same procedures as the SPION- and SPION-conjugate-treated mice. The mice are allotted to the following treatment groups:

| | | Inoculated with Tumor; Tumor | | | | |
|---|---|---|---|---|---|---|
| | | Surgically Removed | | | | |
| | | | | | LHRH + | |
| | Inoculated | no | LHRH | βCG | βCG | No |
| | with Tumor; | pre- | pre- | pre- | pre- | tumor |
| Treatment | Tumor not | treat- | treat- | treat- | treat- | inocu- |
| Group | Removed | ment | ment | ment | ment | lation |
| Saline | 12 | 12 | 12 | 12 | | 12 |
| SPION | 12 | 12 | 12 | 12 | | 12 |
| (250 | | | | | | |
| mg/kg) | | | | | | |
| βCG- | 12 | 12 | | 12 | 12 | 12 |
| SPION | | | | | | |
| (250 | | | | | | |
| mg/kg) | | | | | | |
| LHRH- | 12 | 12 | 12 | | 12 | 12 |
| SPION | | | | | | |
| (250 | | | | | | |
| mg/kg) | | | | | | |
| LHRH/βCG- | 12 | 12 | 12 | 12 | 12 | 12 |
| SPION | | | | | | |
| (250 | | | | | | |
| mg/kg) | | | | | | |

The experimental procedures are otherwise as described for Example 17.

Predicted Results. We have now shown that the ligand-conjugated SPIONs have bound to and been incorporated by both tumors and metastatic cells. We predict that groups pretreated with ligand (βCG or LHRH) should accumulate less iron than groups not pretreated with ligand, because the free ligand will occupy some of the receptor sites on the cell membranes. We also predict that ligand-bound SPIONs will be incorporated into metastases at a higher rate when the primary tumor is removed, because the metastasis load should then be higher. The double-ligand SPIONs may accumulate in higher concentrations in the cancer cells than the single ligand-bearing SPIONs. Only low levels of uptake are expected into tumor and metastases in test groups treated with unconjugated SPIONs, comparable to uptake levels seen in normal tissues. Mice without tumors, and tumor-bearing mice with SPION-only injections are expected to have similar iron accumulation patterns in peripheral organs.

This experiment will also determine whether single-seeded cells, which have not yet developed into vascularized secondary tumors, can be successfully targeted with SPION conjugates.

### EXAMPLE 19

Minimum Time for Optimal Iron Accumulation in Metastases. Routine dose-response tests will be conducted to determine optimal dosages to ad minister the ligand-conjugated SPIONS. To determine the shortest time for maximal iron accumulation in metastases, 120 female nude mice are inoculated with MDA-MB-435S.luc cells as described above. Tumors are surgically removed 25 days after propagation. Treatment is conducted 39 days after tumor cell injection. Mice are injected with saline or SPION, βCG-SPION, LHRH−SPION, or LHRH/βCG-SPION (250 mg/kg per injection). The mice are allotted into 10 groups of 12 mice each, which are imaged by MRI 1 h, 4 h, 8 h, 24 h, and 48 h, after the respective injections, and then sacrificed. The animals are necropsied, and tissues analyzed as described above to determine the time course of iron accumulation, and the minimal time for optimal accumulation.

### EXAMPLE 20

Optimal Concentration to Enhance MRI Sensitivity. This experiment is similar to the prior experiment, except that MRI sensitivity is assessed, rather than iron accumulation per se, to determine the minimal time to acquire optimal MRI sensitivity. The relationship of magnetic particle concentration to relaxation time is biphasic. As concentration increases, the relaxation time first increases, then peaks, and then declines to zero at a critical concentration. Above the critical concentration the relaxation time increases again. The critical concentration, and the shape of this biphasic curve will vary depending on factors such as the type of cell and tissue. We predict that resolution limits in MRI images could be as fine as 100 to 1000 microns; compared to the resolution of several millimeters that is currently possible with commercially available Gd-based contrast agents. Gd has a higher toxicity, and a faster excretion rate. A resolution of 100 microns allows single cells having diameter between 10 and 100 microns to be detected in vivo.

### EXAMPLE 21

Pharmacokinetics of iron nanoparticles. To determine the pharmacokinetic profile of SPIONs and their conjugates, 4 groups of tumor-xenograft-bearing mice (n=10) are injected with the various concentrations of conjugated SPIONs as determined above. A fifth group serves as saline control. The mice are housed for 2, 10, 30, and 60 days after injection, and are then sacrificed to determine iron content in peripheral tissues. This experiment uses 50 female nude mice, and will establish the excretion profile of LHRH−SPIONs over a period of 60 days. These data will show how the distribution pattern changes over time, and whether the nanoparticles are simply excreted or are stored in the target tissues. We predict that iron content in kidney, liver, and spleen will decrease rather rapidly with time, due to normal excretion processes. We expect a slower depletion of iron from tumor and metastatic tissues.

### EXAMPLE 22

Resolution Limits for MRI Detection of Cancer in vivo. The tumor size data and MR images are compared to establish the resolution limits of the MRI imaging methods using the conjugated SPIONs. The resolution limit is taken to correspond to the smallest number of cancer cells (as determined by TEM analysis) corresponding to MRI-detectable images at tumor or metastasis sites. In cases where the TEM analyses confirm nanoparticle accumulation, the angular dependence of the iMQC imaging is determined for the volumes in which the tumor sites are expected. This should provide subvoxel structural information on a scale that is smaller than the diffusion tensor.

### EXAMPLE 23

The experimental results obtained in rodents are confirmed in additional trials in non-rodent, non-human mammals (e.g., dog, monkey) prior to commencing clinical trials in humans. All trials, both in non-human and in human subjects, are conducted in accordance with applicable laws and regulations.

### EXAMPLE 24

In vivo magnetic resonance imaging of human patients is conducted. Patients with breast cancer or prostate cancer are divided into different treatment groups: LHRH-nanoparticles or saline. The patients undergo magnetic resonance imaging both before and 20-48 hours after infusion of the nanoparticles, using T2 weighted FSE (4000/119) imaging. Different groups of six patients each are injected intravenously, intraarterially, or subcutaneously. Dosage is 1-3.4 mg/kg, suspension in saline, infusion over a period of 30 minutes, or other rate as suggested by the results of the experiments in laboratory animals. After 1, 4, and 24 hours the patients are examined by MRI at 0.5-3.0 Tesla. These experiments will determine optimal dosage and route of administration, which will likely vary for different types of cancers. Cancers other than breast and prostate will be the subject of similar testing to determine optimal dosage and route of administration, whenever a receptor is preferentially expressed in tumor tissue. Cancers and their metastases that may be targeted with LHRH- or βCG-conjugated particles include pancreatic, lung, ovarian, melanoma, prostate, breast, uterine, testicular, and bladder, as well as metastases of these or the other cancer types described in this specification. Other ligands that may be used in practicing this invention include, for example, LHRH (pancreatic, prostate, breast, endometrial, colon, ovarian, non-Hodgkin's lymphoma, melanoma, brain, oral, hepatic, renal, and lung cancers), Her2/neu (breast and prostate cancers), transferrin (colon, bladder, and many other cancers), folate (lung, kidney, colon cancers), MSH (melanoma), EGF, estradiol (gonadal cancers), testosterone (gonadal cancers), FSH (gonadal cancers), progesterone (gonadal cancers), LH, anti-CD20, anti-CD8, anti-CD34, anti-Her-2, anti-CD33, αᵥβ₃ somatostatin, growth hormone, glucagon-like peptide (GLP), pituitary adenylate cyclase activating peptide (PACAP), growth hormone releasing hormone (GHRH) (colon, pancreatic, and non-small cell lung cancer), and bombesin; as well as analogs or agonists or antagonists of the above ligands, and fragments and modifications of the above ligands, such as LHRH agonists and antagonists, or GHRH agonists and antagonists.

Mixtures of ligands may also be used on the surface of the nanoparticles, and may have synergistic advantages in certain cases. For example, LHRH may be used in conjunction with transferrin or folate. The transferrin or folate targets cancer cells such as colon, bladder, lung, or kidney as discussed above, and the LHRH inhibits RES uptake of the particles.

Antibody fragments may also be used to label particles; however, cellular uptake will be slower than with receptor-mediated endocytosis. Antibody-labeled particles may be used in this invention for imaging, although their cellular uptake may be less efficient. Cardiovascular tissues may be imaged by using αᵥβ₃; as the ligand. Tissues undergoing inflammation may be imaged using vasoactive intestinal peptide as the ligand.

### EXAMPLE 25

Solid tumors require the development of new blood vessels for growth beyond about 2 mm, a process known as angiogenesis. The new blood vessels feed and nourish the tumor and allow tumor cells to escape into the circulation and to lodge in other organs (tumor metastases). Angiogenesis is difficult to visualize with current MRI techniques. The present technique may be used to image angiogenic vessels in vivo, using conjugates in which the ligands are specific to angiogenic vessels. One such ligand is the cyclic peptide asparagine-glycine-arginine (cNGR), which is specific for the aminopeptidase CD13, a protein that is over-expressed by angiogenic endothelial cells. See A. Dirksen et al., “A supramolecular approach to multivalent target-specific MRI contrast agents for angiogenesis,” Chem. Commun., pp. 2811-2813 (2005).

### EXAMPLE 26

In vitro testing on macrophage uptake of the nanoparticles showed that the LHRH−SPIONs were incorporated by human macrophages significantly less than free SPIONs. At an iron concentration of 20 μg/mL, only about 5% of LHRH−SPIONs were taken up by macrophages, compared to 86% for SPIONs, a thirteen-fold difference (P<0.004). (data not shown). A reduction of LHRH-ligand on the surface of the nanoparticles increased macrophage uptake due to a reduced coating effect.

### EXAMPLE 27

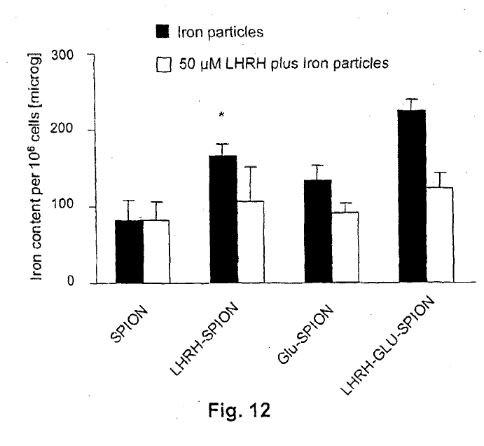

The effect of a spacer molecule on cellular uptake was tested in vitro on MDA-MB-435S.luc breast cancer cells, which express LHRH receptors. Iron incubation was conducted at 1 mg/ml for different conditions for 3 hours. Accumulation in the breast cancer cells was 82.3±25 pg/cell for SPIONs, 165±16 pg/cell for LHRH−SPIONs, and 223±16 pg/cell with LHRH-Glu-SPION. By contrast, in the presence of LHRH, accumulation was significantly reduced for LHRH−SPIONs (106±26 pg/cell) and for LHRH-Glu-SPIONs (123±25 pg/cell), p<0.001 in both cases. These observations support our hypothesis that iron uptake was driven by receptor-mediated endocytosis. Surprisingly, the introduction of a spacer significantly increased intracellular iron uptake from 165 to 223.3, p<0.001. See FIG. 12.

### EXAMPLE 28

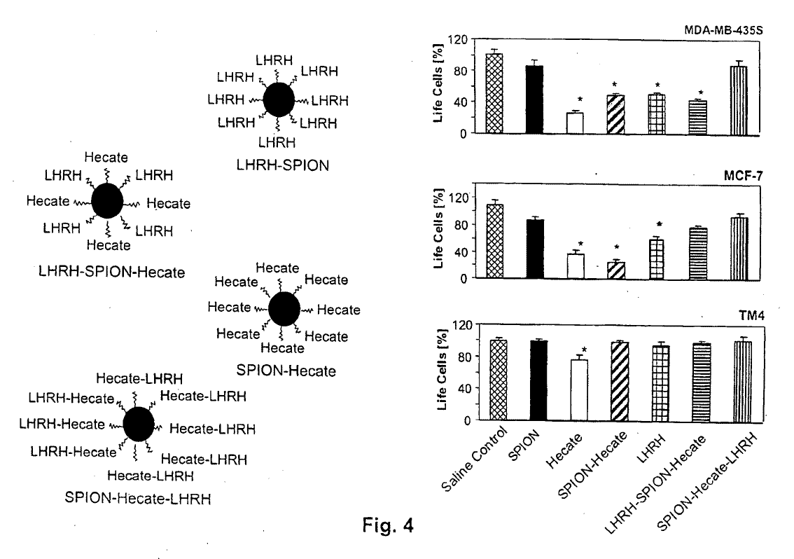

Pancreatic, prostate, breast, and lung cancers may be targeted by LHRH, linked to a toxin or drug such as a lytic peptide and to a SPION. In one study we tested the use of LHRH−SPIONs conjugated to a toxin or drug for both treatment and imaging of tumors and metastases. A particle with a ligand and a toxin (drug) may be made in at least three different configurations: ligand-toxin(drug)-SPION, ligand-SPION-toxin(drug), or toxin(drug)-ligand-SPION. For example, a novel particle comprising a SPION conjugated to the membrane-disrupting (or “lytic”) peptide hecate and to LHRH may be made in at least the following three configurations: LHRH−SPION−Hecate (alternating decoration of SPION surface), SPION−Hecate-LHRH (lytic peptide moiety bound to LHRH and SPION at the same time), or SPION-LHRH-Hecate (LHRH bound to lytic peptide and SPION at the same time). The specificity and potency of the first two of these constructs (LHRH−SPION−Hecate and SPION−Hecate-LHRH) were tested in vitro in LHRH-receptor-expressing breast cancer cell lines (MCF-7 and MDA-MB-435S.luc), as well as in the mouse Sertoli cell line (TM4), which does not express LHRH receptors. The SPIONs with alternating decoration of Hecate and LHRH (LHRH−SPION−Hecate) killed 60-80% of MCF-7 and MDA-MB-435S.luc cells at a concentration of 10 μM after 2 hours; no toxicity was observed in the TM4 cells. The constructs SPION−Hecate-LHRH and LHRH−SPION were not toxic. (The potential construct SPION-LHRH-Hecate was not tested in these experiments.) See FIG. 4. EC₅₀ (μM) values for MDA-MB-435S.luc cells were 9.7±1.6 for LHRH-Hecate, 17.2±2.4 for SPION−Hecate (p<0.01), and 22.3±2.9 for LHRH+SPION+Hecate (P<0.001). These data suggested that hecate required direct contact for optimal membrane interaction. Based on the results reported above, the introduction of a spacer is expected to improve ligand-receptor interaction; and, in the case of a lytic peptide, to improve the peptide-membrane interaction. The spacer may also improve plasma stability of the particles.

### EXAMPLE 29

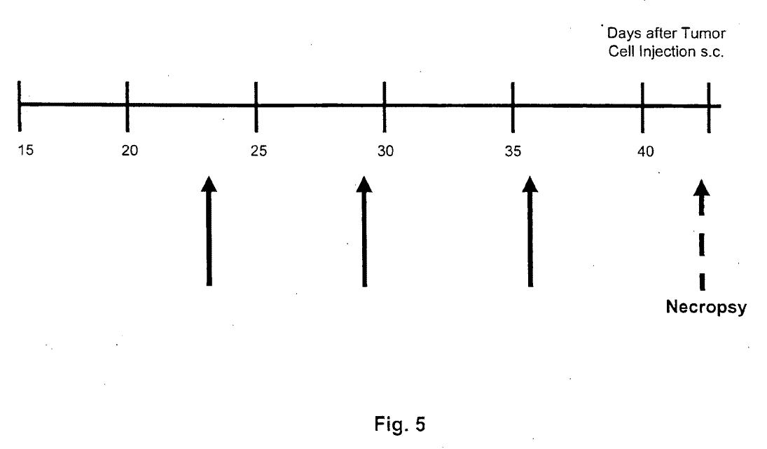

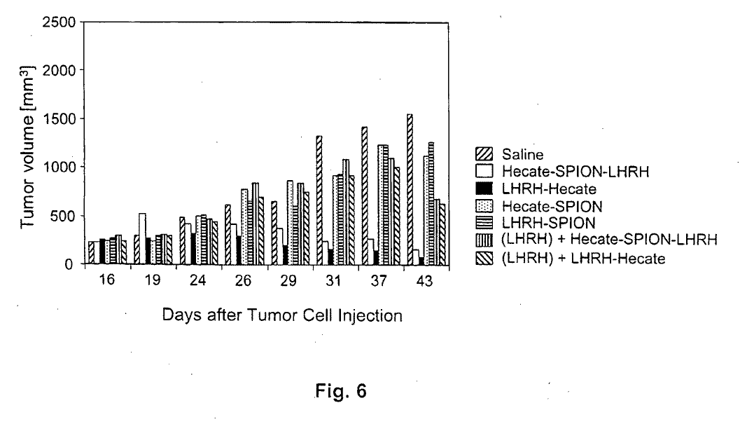

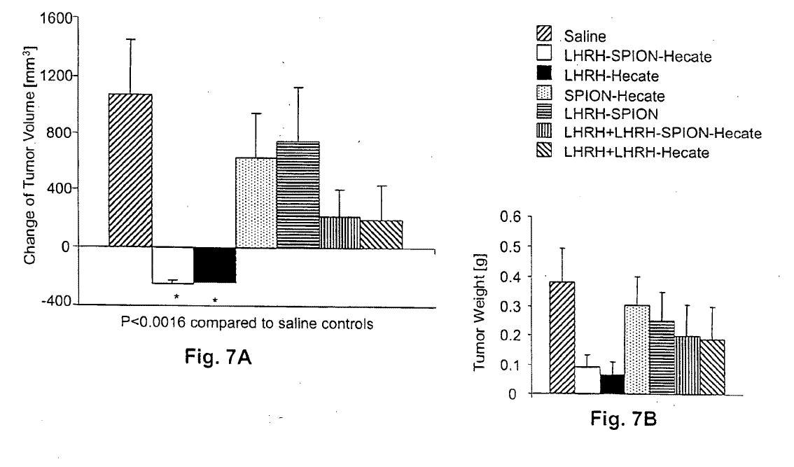

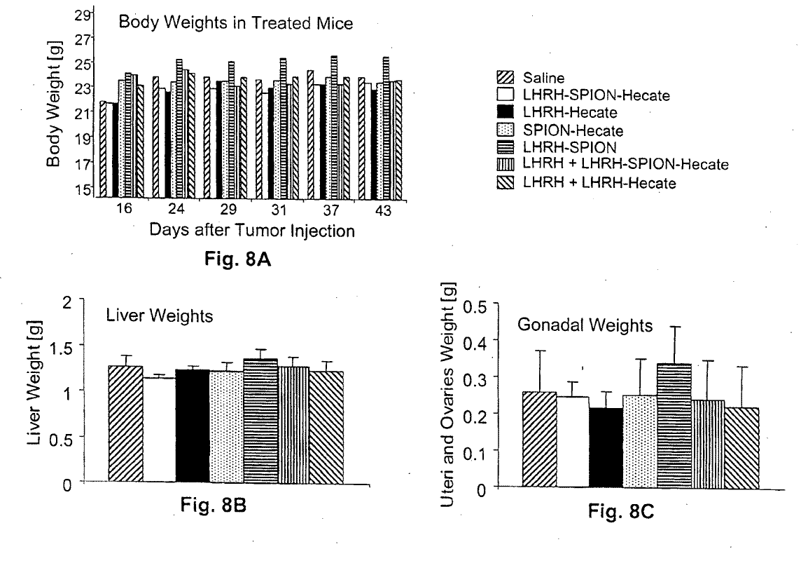

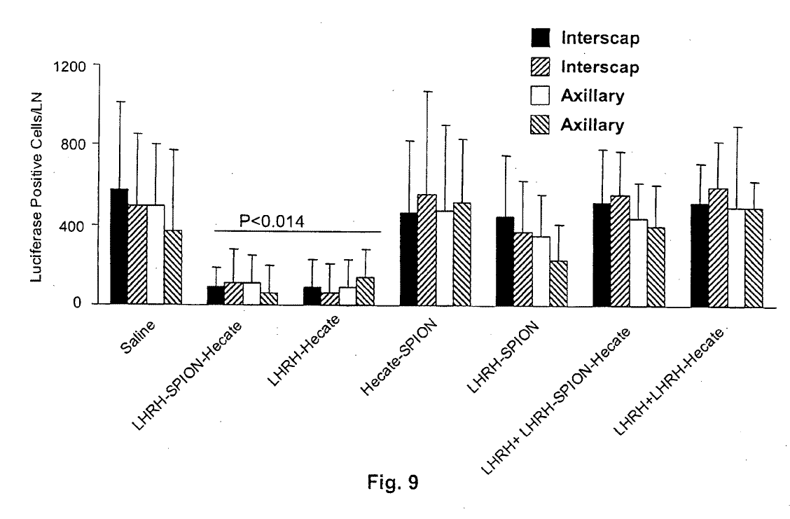

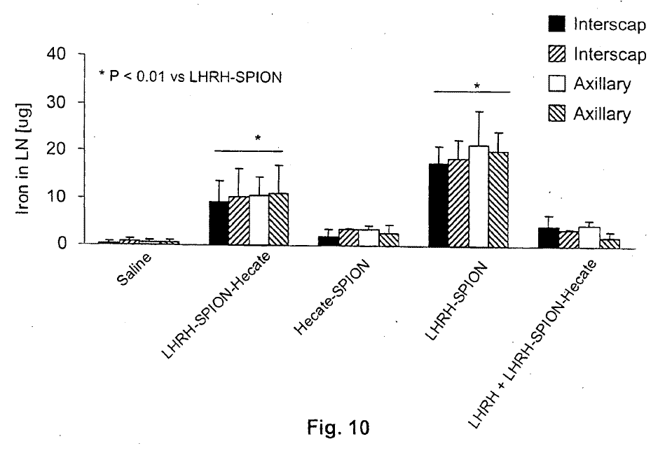

MDA-MB-435S.luc tumor bearing mice were treated at 3 weeks, 4 weeks, and 5 weeks post-xenografts, by injections into the lateral tail vein, with SPION−Hecate (7.5 mg/kg), or LHRH−SPION−Hecate, or LHRH-Hecate, or Hecate (8 mg/kg) (all dosages based on lytic peptide content) (N=8). Mice were sacrificed and necropsied 6 weeks post-xenografts. See FIG. 5. In the LHRH-Hecate and LHRH−SPION−Hecate groups, tumor volumes were arrested at the start of treatment and then diminished significantly during the following 28 days. Tumor weights at necropsy, 28 days after the start of treatment, were as follows:
0.37±0.1 g saline controls
0.3±0.1 SPION+Hecate (p<0.16)
0.09±0.04 LHRH−SPION−Hecate (p<0.0016)
0.07±0.04 LHRH-Hecate (p<0.0016)
0.24±0.09 LHRH−SPION (p<0.05)
   In a parallel set of experiments under otherwise similar conditions, no treatment response was observed in groups that had been pre-treated with LHRH, suggesting that the LHRH in these experiments occupied the LHRH receptors on the target cells. See FIGS. 6 and 7. Similar patterns were observed for reduction in volume for lung metastases, bone metastases, and lymph node metastases in MDA-MB-435S.luc tumor bearing mice. Bodyweights, liver weights, and gonadal weights remained stable during all treatments. See FIGS. 8 and 9. The breast cancer tumors and metastases were specifically targeted and destroyed by LHRH-conjugated SPIONs carrying a toxin (hecate). The same particles may be used for simultaneous treatment and imaging, e.g., for directly monitoring treatment response in cancer patients. We observed that iron accumulation in lymph nodes of mice treated with LHRH−SPION−hecate was comparable to that in mice treated with LHRH−SPION. See FIG. 10. By contrast, Hecate-SPION did not destroy or accumulate in tumors or metastases.

LHRH-hecate was used as for comparison to monitor the efficacy of the new construct, as exemplified by LHRH−SPION−hecate. From previously published experiments it is known that LHRH-hecate is effective in killing cancer cells, both in vitro and in vivo. The above data confirmed our hypothesis that LHRH−SPION−Hecate also kills cancer cells, and in addition has the advantage of facilitating images to monitor the progress of treatment. The treated organ retains the SPIONs for a time, and may be imaged both during and following treatment.

Without wishing to be bound by this theory, we hypothesize that it may be possible that retained drug within a treated organ may continue to be active against any remaining tumor cells for a time. Imaging is conducted to determine morphological changes in the treated tissue. We expect the imaging to show a confined, structured accumulation of iron oxide particles in intact tumors, along with a more diffuse pattern of iron oxide particles in the destroyed tissue. For example, we would expect apoptosis-inducing drugs to destroy cancer cells slowly, and therefore imaging would be expected to differ from what would be seen with a fast-acting, necrosis-inducing compound. Anti-angiogenesis compounds would be expected to generate still different images, and so forth. The invention may be used to facilitate detection of a tumor cell cluster, which we would expect to be imaged as a confined entity initially, and then either to disintegrate or to diffuse as tumor cells disintegrate.

Linking Toxins or Drugs, Spacers, and Ligands to SPIONS

The chemistry for linking toxin (or drug) directly to the iron oxide nanoparticles is essentially the same for the toxin (or drug) and the ligand, and is based on amide bond formation via carbodiimide reaction. Essentially any anticancer agent or targeting agent with a free carboxyl group, for example, may be used in a carbodiimide reaction. If a particular agent otherwise lacks a carboxyl group, a carboxyl group may be incorporated into the agent through any of a number of routes known in organic chemistry. Alternatively, one may use a thiol group to bind drugs or targeting agents to a gold shell surrounding an iron or iron oxide nanoparticle core. The (Ligand)ₓ-Nanoparticle-Drug_{y} construct may contain more than one type of ligand, more than one type of drug molecule, or both (x and y are variables). Other linking moieties known in the art may also be used.

Toxins Suitable for Use in the Present Invention

Any of a number of toxins may be used in the present invention. A toxin may be of plant, animal, bacterial, fungal, viral, or synthetic origin. Other therapeutic drugs that are not necessarily “toxins” may also be used.

For example, there are many bacterial toxins that use an A/B subunit motif, in which the A subunit is toxic once it enters a cell but has no ability to cross cell membranes unassisted, and in which the B subunit (or multi-subunit complex) binds to cells but has no toxicity on its own. The A subunit, even when injected systemically, is non-toxic. See, e.g., Balfanz et al., 1996; Middlebrook and Dorland, 1984. The A or active subunit may be used in this invention alone, because the particles are endocytosed by cells having appropriate receptors. It will therefore not be necessary to include sequences coding for the B or cell-binding component. The A subunit will kill the cells that endocytose the particles, but will not damage other cells that lack the receptor. Examples include the A subunit of cholera toxin, which destroys ion balance, and the A subunit of diphtheria toxin, which terminates protein synthesis. Other toxins comprise a single peptide chain having separate domains, where one domain functions to enable entry into the cell and a second domain is toxic. Such a multidomain peptide toxin could be truncated to use only the toxin domain. One example of a truncated toxin that has been used in other systems to kill artificially targeted cells is the truncated form of exotoxin A from Pseudomonas aeruginosa (Brinkman et al., 1993, Pastan and FitzGerald, 1991, and Wels et al., 1995) The commonly used ricin toxin from plants also uses this same type of A/B subunit motif. Lee, H. P. et al., “Immunotoxin Therapy for Cancer,” JAMA, vol. 269, pp. 78-81 (1993). The diphtheria toxin A polypeptide has been successfully used (in another context) to selectively kill cell lineages in transgenic mice. See R. Palmiter et al., “Cell lineage ablation in transgenic mice by cell-specific expression of a toxin gene,” Cell, vol. 50, pp. 435-443 (1987).

Toxins (or drugs) that may be used in the present invention include, for example, the following

Alkylating Agents, e.g., cyclophosphamide, melphalon, busolfan, procarbazine.
Antibiotics, e.g., membrane disrupting lytic peptides (discussed at greater length below), daunorubicin, doxorubicin, idarubicin, mitomycin, mitoxanthrone, pentostatin.
Antimetabolites, e.g., fluorouracil, capecitabine, fludarabine, mercaptopurine, gemcitabine.
Hormonal Oncologics, e.g., tamoxifen, leuprolide, topotecan.
Mitosis inhibitors, e.g., etopside.
Antimicrotubule reagents, e.g., vinblastin, vincristine, paclitaxel, docetaxel.
Antisense DNA, DNA delivery, apoptosis promoting compounds, cell cycle interfering compounds, other anti-cancer agents, e.g., p53, p21^{waf1}, bcl2, caspase-6, caspase-3, Bclx_{L}, Ras, cisplatin, oxiplatin, asparaginase, hydroxyurea.

Additional anti-cancer compounds that may be used in practicing this invention include, among others, the following:

Alkylating and Oxidizing Agents

### I. Nitrogen Mustards

mechlorethamine (Mustargen)
cyclophosphamide (Cytoxan, Neosar)
ifosfamide (Ifex)
phenylalanine mustard; melphalan (Alkeran)
chlorambucol (Leukeran)
uracil mustard
estramustine (Emcyt)

### II. Ethylenimines

thiotepa (Thioplex)

### III. Alkyl Sulfonates

busulfan (Myerlan)

### IV. Nitrosureas

lomustine (CeeNU)
carmustine (BiCNU, BCNU)
streptozocin (Zanosar)

### V. Triazenes

dacarbazine (DTIC-Dome)
temozolamide (Temodar)

### VI. Platinum Coordination Complexes

cis-platinum, cisplatin (Platinol, Platinol AQ)
carboplatin (Paraplatin)

### VII. Others

altretamine (Hexylen)
arsenic (Trisenox)

Antimetabolites

### I. Folic Acid Analogs

methotrexate (Amethopterin, Folex, Mexate, Rheumatrex)

### II. Pyrimidine Analogs

5-fluorouracil (Adrucil, Efudex, Fluoroplex)
floxuridine, 5-fluorodeoxyuridine (FUDR)
capecitabine (Xeloda)
fludarabine: (Fludara)
cytosine arabinoside (Cytaribine, Cytosar, ARA-C)

### III. Purine Analogs

6-mercaptopurine (Purinethiol)
6-thioguanine (Thioguanine)
gemcitabine (Gemzar)
cladribine (Leustatin)
deoxycoformycin; pentostatin (Nipent)

Antibiotics

doxorubicin (Adriamycin, Rubex, Doxil, Daunoxome-liposomal preparation)
daunorubicin (Daunomycin, Cerubidine)
idarubicin (Idamycin)
valrubicin (Valstar)
epirubicin
mitoxantrone (Novantrone)
dactinomycin (Actinomycin D, Cosmegen)
mithramycin, plicamycin (Mithracin)
mitomycin C (Mutamycin)
bleomycin (Blenoxane)
procarbazine (Matulane)

Mitotic Inhibitors

### I. Taxanes (Diterpenes)

paclitaxel (Taxol)
docetaxel (Taxotere)

### II. Vinca Alkaloids

vinblatine sulfate (Velban, Velsar, VLB)
vincristine sulfate (Oncovin, Vincasar PFS, Vincrex)
vinorelbine sulfate (Navelbine)

Chromatin Function Inhibitors

### I. Camptothecins

topotecan (Camptosar)
irinotecan (Hycamtin)

### II. Epipodophyllotoxins

etoposide (VP-16, VePesid, Toposar)
teniposide (VM-26, Vumon)

Hormones and Hormone Inhibitors

### I. Estrogens

diethylstilbestrol (Stilbestrol, Stilphostrol)
estradiol, estrogen
esterified estrogens (Estratab, Menest)
estramustine (Emcyt)

### II. Antiestrogens

tamoxifen (Nolvadex)
toremifene (Fareston)

### III. Aromatase Inhibitors

anastrozole (Arimidex)
letrozole (Femara)

### IV. PROGESTINS

17-OH-progesterone
medroxyprogesterone
megestrol acetate (Megace)

### V. LHRH Agonists and Antagonists

goserelin (Zoladex)
leuprolide (Leupron)

### Cetrorelix (Cetrotide)

ganerelix (Antagon)

### VI. Androgens

testosterone
methyltestosterone
fluoxmesterone (Android-F, Halotestin)

### VII. Antiandrogens

flutamide (Eulexin)
bicalutamide (Casodex)
nilutamide (Nilandron)

### VIII. Inhibitors of Steroid Synthesis

aminoglutethimide (Cytadren)
ketoconazole (Nizoral)

Antibodies

rituximab (Rituxan)
trastuzumab (Herceptin)
gemtuzumab ozogamicin (Mylotarg)
tositumomab (Bexxar)
bevacizumab

Immunomodulators

denileukin diftitox (Ontak)
levamisole (Ergamisol)
bacillus Calmette-Guerin, BCG (TheraCys, TICE BCG)
interferon alpha-2a, alpha 2b (Roferon-A, Intron A)
interleukin-2, aldesleukin (ProLeukin)

Angiogenesis Inhibitors

thalidomide (Thalomid)
angiostatin
endostatin

Miscellaneous

imatinib mesylate; STI-571 (Gleevec)

### I-aspariginase (Elspar, Kidrolase)

pegaspasgase (Oncaspar)
hydroxyurea (Hydrea, Doxia)
leucovorin (Welicovorin)
mitotane (Lysodren)
porfimer (Photofrin)
tretinoin (Veasnoid)

Lytic Peptides Useful in the Present Invention.

Preferred toxins for use in destroying cancer cells in the present invention are the so-called lytic peptides. “Lytic peptides,” or “antimicrobial amphipathic peptides,” are relatively small, generally containing 20 to 50 amino acids (or even fewer), and are capable of forming an amphipathic alpha helix in a hydrophobic environment, wherein at least part of one face is predominantly hydrophobic and at least part of the other face is predominately hydrophilic and is positively charged at physiological pH. Such structures can be predicted by applying the amino acid sequence to the Edmundson helical wheel (Schiffer and Edmundson, 1967). In addition to their small size, such peptides are widely distributed in nature and vary significantly in toxicity. They can also be designed to possess different levels of lytic activity. Many of these toxins are inactivated by serum factors, and cause systemic tissue damage only when present in high concentrations. Typically, when applied to cells in culture, a few micrograms per mL are required to kill the cultured cells. The level of toxicity of lytic peptides is determined by the amino acid composition and sequence. Different peptides can have widely differing levels of toxicity, to be chosen as needed for a particular use.

Lytic peptides are small, basic peptides. Native lytic peptides appear to be major components of the antimicrobial defense systems of a number of animal species, including those of insects, amphibians, and mammals. They typically comprise 23-39 amino acids, although they can be smaller. They have the potential for forming amphipathic alpha-helices. See Boman et al., “Humoral immunity in Cecropia pupae,” Curr. Top. Microbiol. Immunol. vol. 94/95, pp. 75-91 (1981); Boman et al., “Cell-free immunity in insects,” Ann. Rev. Microbiol., vol. 41, pp. 103-126 (1987); Zasloff, “Magainins, a class of antimicrobial peptides from Xenopus skin: isolation, characterization of two active forms, and partial DNA sequence of a precursor,” Proc. Natl. Acad. Sci. USA, vol. 84, pp. 3628-3632 (1987); Ganz et al., “Defensins natural peptide antibiotics of human neutrophils,” J. Clin. Invest., vol. 76, pp. 1427-1435 (1985); and Lee et al., “Antibacterial peptides from pig intestine: isolation of a mammalian cecropin,” Proc. Natl. Acad. Sci. USA, vol. 86, pp. 9159-9162 (1989).

Known amino acid sequences for lytic peptides may be modified to create new peptides that would also be expected to have lytic activity by substitutions of amino acid residues that preserve the amphipathic nature of the peptides (e.g., replacing a polar residue with another polar residue, or a non-polar residue with another non-polar residue, etc.); by substitutions that preserve the charge distribution (e.g., replacing an acidic residue with another acidic residue, or a basic residue with another basic residue, etc.); or by lengthening or shortening the amino acid sequence while preserving its amphipathic character or its charge distribution. Lytic peptides and their sequences are disclosed in Yamada et al., “Production of recombinant sarcotoxin IA in Bombyx mori cells,” Biochem. J., vol. 272, pp. 633-666 (1990); Taniai et al., “Isolation and nucleotide sequence of cecropin B cDNA clones from the silkworm, Bombyx mori,” Biochimica Et Biophysica Acta, vol. 1132, pp. 203-206 (1992); Boman et al., “Antibacterial and antimalarial properties of peptides that are cecropin-melittin hybrids,” FEBS Letters, vol. 259, pp. 103-106 (1989); Tessier et al., “Enhanced secretion from insect cells of a foreign protein fused to the honeybee melittin signal peptide,” Gene, vol. 98, pp. 177-183 (1991); Blondelle et al., “Hemolytic and antimicrobial activities of the twenty-four individual omission analogs of melittin,” Biochemistry, vol. 30, pp. 4671-4678 (1991); Andreu et al., “Shortened cecropin A-melittin hybrids. Significant size reduction retains potent antibiotic activity,” FEBS Letters, vol. 296, pp. 190-194 (1992); Macias et al., “Bactericidal activity of magainin 2: use of lipopolysaccharide mutants,” Can. J. Microbiol., vol. 36, pp. 582-584 (1990); Rana et al., “Interactions between magainin-2 and Salmonella typhimurium outer membranes: effect of Lipopolysaccharide structure,” Biochemistry, vol. 30, pp. 5858-5866 (1991); Diamond et al., “Airway epithelial cells are the site of expression of a mammalian antimicrobial peptide gene,” Proc. Natl. Acad. Sci. USA, vol. 90, pp. 4596 ff (1993); Selsted et al., “Purification, primary structures and antibacterial activities of β-defensins, a new family of antimicrobial peptides from bovine neutrophils,” J. Biol. Chem., vol. 268, pp. 6641 ff (1993); Tang et al., “Characterization of the disulfide motif in BNBD-12, an antimicrobial β-defensin peptide from bovine neutrophils,” J. Biol. Chem., vol. 268, pp. 6649 ff (1993); Lehrer et al., Blood, vol. 76, pp. 2169-2181 (1990); Ganz et al., Sem. Resp. Infect. I., pp. 107-117 (1986); Kagan et al., Proc. Natl. Acad. Sci. USA, vol. 87, pp. 210-214 (1990); Wade et al., Proc. Natl. Acad. Sci. USA, vol. 87, pp. 4761-4765 (1990); Romeo et al., J. Biol. Chem., vol. 263, pp. 9573-9575 (1988); Jaynes et al., “Therapeutic Antimicrobial Polypeptides, Their Use and Methods for Preparation,” WO 89/00199 (1989); Jaynes, “Lytic Peptides, Use for Growth, Infection and Cancer,” WO 90/12866 (1990); Berkowitz, “Prophylaxis and Treatment of Adverse Oral Conditions with Biologically Active Peptides,” WO 93/01723 (1993).

Families of naturally-occurring lytic peptides include the cecropins, the defensins, the sarcotoxins, the melittins, and the magainins. Boman and coworkers in Sweden performed the original work on the humoral defense system of Hyalophora cecropia, the giant silk moth, to protect itself from bacterial infection. See Hultmark et al., “Insect immunity. Purification of three inducible bactericidal proteins from hemolymph of immunized pupae of Hyalophora cecropia,” Eur. J. Biochem., vol. 106, pp. 7-16 (1980); and Hultmark et al., “Insect immunity. Isolation and structure of cecropin D. and four minor antibacterial components from cecropia pupae,” Eur. J. Biochem., vol. 127, pp. 207-217 (1982).

Infection in H. cecropia induces the synthesis of specialized proteins capable of disrupting bacterial cell membranes, resulting in lysis and cell death. Among these specialized proteins are those known collectively as cecropins. The principal cecropins—cecropin A, cecropin B, and cecropin D—are small, highly homologous, basic peptides. In collaboration with Merrifield, Boman's group showed that the amino-terminal half of the various cecropins contains a sequence that will form an amphipathic alpha-helix. Andrequ et al., “N-terminal analogues of cecropin A: synthesis, antibacterial activity, and conformational properties,” Biochem., vol. 24, pp. 1683-1688 (1985). The carboxy-terminal half of the peptide comprises a hydrophobic tail. See also Boman et al., “Cell-free immunity in Cecropia,” Eur. J. Biochem., vol. 201, pp. 23-31 (1991).

A cecropin-like peptide has been isolated from porcine intestine. Lee et al., “Antibacterial peptides from pig intestine: isolation of a mammalian cecropin,” Proc. Natl. Acad. Sci. USA, vol. 86, pp. 9159-9162 (1989).

Cecropin peptides have been observed to kill a number of animal pathogens other than bacteria. See Jaynes et al., “In Vitro Cytocidal Effect of Novel Lytic Peptides on Plasmodium falciparum and Trypanosoma cruzi,” FASEB, 2878-2883 (1988); Arrowood et al., “Hemolytic properties of lytic peptides active against the sporozoites of Cryptosporidium parvum,” J. Protozool., vol. 38, No. 6, pp. 161S-163S (1991); and Arrowood et al., “In vitro activities of lytic peptides against the sporozoites of Cryptosporidium parvum,” Antimicrob. Agents Chemother., vol. 35, pp. 224-227 (1991). However, normal mammalian cells do not appear to be adversely affected by cecropins, even at high concentrations. See Jaynes et al., “In vitro effect of lytic peptides on normal and transformed mammalian cell lines,” Peptide Research, vol. 2, No. 2, pp. 1-5 (1989); and Reed et al., “Enhanced in vitro growth of murine fibroblast cells and preimplantation embryos cultured in medium supplemented with an amphipathic peptide,” Mol. Reprod. Devel., vol. 31, No. 2, pp. 106-113 (1992).

Defensins, originally found in mammals, are small peptides containing six to eight cysteine residues. Ganz et al., “Defensins natural peptide antibiotics of human neutrophils,” J. Clin. Invest., vol. 76, pp. 1427-1435 (1985). Extracts from normal human neutrophils contain three defensin peptides: human neutrophil peptides HNP-1, HNP-2, and HNP-3. Defensin peptides have also been described in insects and higher plants. Dimarcq et al., “Insect immunity: expression of the two major inducible antibacterial peptides, defensin and diptericin, in Phormia terranvae,” EMBO J., vol. 9, pp. 2507-2515 (1990); Fisher et al., Proc. Natl. Acad. Sci. USA, vol. 84, pp. 3628-3632 (1987).

Slightly larger peptides called sarcotoxins have been purified from the fleshfly Sarcophaga peregrina. Okada et al., “Primary structure of sarcotoxin I, an antibacterial protein induced in the hemolymph of Sarcophaga peregrina (flesh fly) larvae,” J. Biol. Chem., vol. 260, pp. 7174-7177 (1985). Although highly divergent from the cecropins and defensins, the sarcotoxins presumably have a similar antibiotic function.

Other lytic peptides have been found in amphibians. Gibson and collaborators isolated two peptides from the African clawed frog, Xenopus laevis, peptides which they named PGS and Gly¹⁰Lys²²PGS. Gibson et al., “Novel peptide fragments originating from PGLₐ and the caervlein and xenopsin precursors from Xenopus laevis,” J. Biol. Chem., vol. 261, pp. 5341-5349 (1986); and Givannini et al., “Biosynthesis and degradation of peptides derived from Xenopus laevis prohormones,” Biochem. J., vol. 243, pp. 113-120 (1987). Zasloff showed that the Xenopus-derived peptides have antimicrobial activity, and renamed them magainins. Zasloff, “Magainins, a class of antimicrobial peptides from Xenopus skin: isolation, characterization of two active forms, and partial DNA sequence of a precursor,” Proc. Natl. Acad. Sci. USA, vol. 84, pp. 3628-3632 (1987).

Synthesis of nonhomologous analogs of different classes of lytic peptides has been reported to reveal that a positively charged, amphipathic sequence containing at least 20 amino acids appeared to be a requirement for lytic activity in some classes of peptides. Shiba et al., “Structure-activity relationship of Lepidopteran, a self-defense peptide of Bombyx more,” Tetrahedron, vol. 44, No. 3, pp. 787-803 (1988). Other work has shown that smaller peptides can also be lytic.

Cecropins have been shown to target pathogens or compromised cells selectively, without affecting normal host cells. The synthetic lytic peptide known as S-1 (or Shiva 1) has been shown to destroy intracellular Brucella abortus-, Trypanosoma cruzi-, Cryptosporidium parvum-, and infectious bovine herpes virus I (IBR)-infected host cells, with little or no toxic effects on noninfected mammalian cells. See Jaynes et al., “In vitro effect of lytic peptides on normal and transformed mammalian cell lines,” Peptide Research, vol. 2, No. 2, pp. 1-5 (1989); Wood et al., “Toxicity of a Novel Antimicrobial Agent to Cattle and Hamster cells In vitro,” Proc. Ann. Amer. Soc. Anim. Sci., Utah State University, Logan, UT. J. Anim. Sci. (Suppl. 1), vol. 65, p. 380 (1987); Arrowood et al., “Hemolytic properties of lytic peptides active against the sporozoites of Cryptosporidium parvum,” J. Protozool., vol. 38, No. 6, pp. 161S-163S (1991); Arrowood et al., “In vitro activities of lytic peptides against the sporozoites of Cryptosporidium parvum,” Antimicrob. Agents Chemother., vol. 35, pp. 224-227 (1991); and Reed et al., “Enhanced in vitro growth of murine fibroblast cells and preimplantation embryos cultured in medium supplemented with an amphipathic peptide,” Mol. Reprod. Devel., vol. 31, No. 2, pp. 106-113 (1992).

Morvan et al., “In vitro activity of the antimicrobial peptide magainin 1 against Bonamia ostreae, the intrahemocytic parasite of the flat oyster Ostrea edulis,” Mol. Mar. Biol., vol. 3, pp. 327-333 (1994) reports the in vitro use of a magainin to selectively reduce the viability of the parasite Bonamia ostreae at doses that did not affect cells of the flat oyster Ostrea edulis.

Other useful lytic peptides include the “Phor” peptides of M. McLaughlin et al., such as Phor14 and Phor21.

Also of interest are the synthetic peptides disclosed U.S. Pat. Nos. 5,789,542 and 6,566,334, peptides that have lytic activity with as few as 10-14 amino acid residues.

### Miscellaneous

Magnetic nanoparticles in accordance with the present invention may be administered to a patient by any suitable means, including oral, intravenous, parenteral, subcutaneous, intrapulmonary, intranasal administration, or inhalation. The means of administration may depend on the type of cancer being imaged. For example, inhalation might be well suited for detecting lung cancers and metastases in the lungs. Intravenous administration will generally be preferred for detecting metastases in various organs, including the brain.

Pharmaceutically acceptable carrier preparations include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, aqueous solutions, emulsions or suspensions, including saline and buffered media. Parenteral vehicles include sodium chloride solution, Ringer's dextrose, dextrose and sodium chloride, lactated Ringer's, or fixed oils. The nanoparticles may be mixed with excipients that are pharmaceutically acceptable and are compatible with the nanoparticles. Suitable excipients include water, saline, dextrose, and glycerol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like. A preferred carrier is phosphate-buffered saline.

The form may vary depending upon the route of administration. For example, compositions for injection may be provided in the form of an ampule, each containing a unit dose amount, or in the form of a container containing multiple doses. For clinical use, it is preferred to aliquot the product in lyophilized form, suitable for reconstitution in saline, for preservation and sterility.

The ligand component of the nanoparticles is preferably stored in lyophilized form, and then reconstituted prior to use. The ligand component of the nanoparticles may optionally be administered or stored in the form of pharmaceutically acceptable salts where such a form may be advantageous for storage or administration. These salts include acid addition salts formed with inorganic acids, for example hydrochloric or phosphoric acid, or organic acids such as acetic, oxalic, or tartaric acid, and the like. Salts also include those formed from inorganic bases such as, for example, sodium, potassium, ammonium, calcium or ferric hydroxides, and organic bases such as isopropylamine, trimethylamine, histidine, procaine and the like.

Initial in vivo animal trials will be conducted in accordance with all applicable laws and regulations, followed by clinical trials in humans in accordance with all applicable laws and regulations.

Definitions. Unless otherwise clearly indicated by context, the following definitions apply in both the specification and claims.

“Nanoparticle(s)” refer to particle(s) having a mean diameter between about 1 nm and about 500 nm or between about 5 nm and about 400 nm, preferably between about 10-150 nm or about 10-100 nm, (Note that the “diameter” of a particle refers to its largest dimension, and does not necessarily imply that the particle has a spherical shape or a circular cross section. The particles may, for example, comprise nanofibers, nanorods, or nanomaterials of other shapes).

The terms “specific,” “site-specific,” “target-specific,” and “targeted” are interchangeable, and refer to particles that preferentially accumulate in a desired tissue by virtue of compounds on the surface of the particles, for example, compounds such as hormones, ligands, receptors, or antibodies, or fragments thereof that selectively bind to receptors, ligands, or epitopes on the surface of cells in that tissue.

The expression “is essentially free of” is the converse of the term “consists essentially of.” A composition is “essentially free of” a component X either if it contains no X at all, or if small amounts of X are present; but in the latter case, the properties of the composition should be substantially the same (in relevant aspects) as the properties of an otherwise identical composition that is free of X. If sufficient X is present that the properties of the composition are substantially altered (in relevant aspects) as compared to the properties of an otherwise identical composition that is free of X, then the composition is not considered to be “essentially free of” component X.

The term “directly bonded” refers to two or more entities (e.g., a ligand and an iron oxide nanoparticle; or a ligand, a spacer, and an iron oxide nanoparticle) that are covalently bonded directly to one another through one or more small linking groups, e.g., an amide group or an ester group. The term “directly bonded” does not encompass bonding of the nanoparticle and ligand via an intermediate coating layer, e.g., a dextran coating. It does encompass bonding via a spacer as discussed above.

A “spacer” is a moiety that covalently links, but places some distance between the nanoparticle surface and the toxin, drug, or ligand. The term “spacer” does not, however, include a coating layer. Preferably the linker is relatively inert after it bonds both to the nanoparticle and to the toxin, drug, or ligand. For example, the conjugate may take the form (nanoparticle)-NH—CO—R—CO-ligand, or (nanoparticle)-NH—CO—R—CO-toxin, or (nanoparticle)-NH—CO—R—CO-drug, or toxin-CO—R—CO—NH(nanoparticle)-NH—CO—R—CO-ligand, or drug-CO—R—CO—NH(nanoparticle)-NH—CO—R—CO-ligand; wherein R may, for example, take the form —(CHX)ₙ, where X is —H or —OH.

The term “effective amount” refers to an amount of the specified nanoparticles that is sufficient to enhance imaging of one or more tumors, metastases., nonvascularized malignant cell clusters, or individual malignant cells to a clinically significant degree; or to an amount of the specified nanoparticles that is sufficient to selectively kill or inhibit one or more tumors, metastases, nonvascularized malignant cell clusters, or individual malignant cells to a clinically significant degree; or an amount that is sufficient to deliver an amount of drug to a targeted tissue in a clinically significant amount; in each case without causing clinically unacceptable side effects on non-targeted tissues.

The term “ligand” should be understood to encompass not only the native ligand, but also analogs of the native ligand. Numerous analogs of many hormones are well known in the art.

Statistical analyses: Unless otherwise indicated, statistical significance is determined by McNemar's test, ANOVA, and the Kruskal-Wallis test for spectrophotometric iron content analysis and luciferase assays. Unless otherwise indicated, statistical significance is determined at the P<0.05 level, or by such other measure of statistical significance as is commonly used in the art for a particular type of determination.

Abbreviations: Some of the abbreviations used in the specification:

### LH Luteinizing Hormone

### LHRH Luteinizing Hormone Releasing Hormone

### CG Chorionic Gonadotropin

βCG Fragment of the beta chain of CG, amino acid residues 81-95

### FSH Follicle Stimulating Hormone

RES Reticulo endothelial system
SPION Superparamagnetic iron oxide nanoparticle

The complete disclosures of all references cited in the specification are hereby incorporated by reference. Also incorporated by reference are the complete disclosures of the following past and future papers, abstracts, presentations, publications, and other materials by the present inventors: C. Leuschner et al., “Targeting breast cancer cells and their metastases through luteinizing hormone releasing hormone (LHRH) receptors using magnetic nanoparticles,” J. Biomed. Nanotech., vol. 2, pp 229-233 (2005); C. Leuschner et al., “Human prostate cancer cells and xenograft are targeted and destroyed through luteinizing hormone releasing hormone receptors,” Prostate, vol. 56, pp. 239-249 (2003); C. Kumar et al., “Functionalized magnetic nanoparticles for an optimized breast cancer drug delivery,” invited talk at 5th International Conference on the Scientific and Clinical Applications of Magnetic Carriers, Lyon, France (May 2004); J. Zhou et al., “Functionalized magnetic nanoparticles for early breast cancer detection,” Mineral, Metals and Materials Society, 134th Annual Meeting, San Francisco (February 2005); J. Zhou et al., “A TEM Study: Biological Distribution of Superparamagnetic Iron Oxide Nanoparticles,” Materials Research Society, San Francisco (March/April 2005); C. Leuschner et al., “Ligand conjugated superparamagnetic iron oxide nanoparticles for early detection of metastases,” NSTI Nanotechnology Conference, Anaheim (May 2005); C. Leuschner et al., “Nanomaterials: Opportunities for Detection of metastatic cancer cells,” 5th LA Conference on Advance Materials and Emerging Technologies, New Orleans (2005); C. Leuschner, “Development of contrast agents for early detection of cancers and metastatic disease,” American Academy for Nanomedicine, Baltimore, Md. (August 2005); C. Leuschner et al., “Targeting breast cancers and metastases with LHRH and a lytic peptide bound to iron oxide nanoparticles,” Clinical Cancer Research, vol. 11 (24), p. 9097S (2005); C. Kumar et al., “Efficacy of lytic peptide bound magnetite nanoparticles in destroying breast cancer cells,” J. Nanoscience and Nanotechnology, vol. 4, pp. 245-249 (2004); C. Leuschner et al., “The use of ligand conjugated superparamagnetic iron oxide nanoparticles (SPION) for early detection of metastases,” NSTI Nanotech. Technical Proceedings, Vol 1, pp. 5-6 (2005); C. Leuschner et al., “Ligand conjugated superparamagnetic iron oxide nanoparticles for early detection of metastases,” paper submitted to Breast Cancer Research Treatment (available online Jun. 3, 2006); J. Zhou et al., “Subcellular accumulation of magnetic nanoparticles in breast tumors and metastases,” Biomaterials, vol. 27, pp. 2001-2008 (2006); C. Leuschner et al., “LHRH-conjugated magnetic iron oxide nanoparticles for detection of breast cancer metastases,” Breast Cancer Research and Treatment (available online Jun. 3, 2006); C. Leuschner et al., “Targeting breast cancer cells and their metastases through luteinizing hormone releasing hormone (LHRH) receptors using magnetic nanoparticles,” J. Biomed. Nanotech., vol. 1, pp. 229-233 (2005); J. Zhou et al., “Sub-cellular accumulation of magnetic nanoparticles in breast tumors and metastases, Biomaterials, vol. 27, pp. 2001-2008 (2006); J. Meng et al., “LHRH-Functionalized Magnetite Nanotargets for Contrast Enhancement of Breast Tumor MRI,” submitted to J. Appl. Phys. (2006). In the event of an otherwise irreconcilable conflict, the present specification shall control.

We claim:

1. A particle comprising:
(a) an iron oxide nanoparticle, wherein the diameter of said iron oxide nanoparticle is between about 1 nm and about 500 nm; and
(b) a plurality of ligand molecules having specific affinity for a selected receptor on mammalian cells; wherein the receptor is adapted to mediate endocytosis; wherein said ligand molecules are directly bonded covalently to said iron oxide nanoparticle; and wherein the plurality of ligand molecules may be the same or different.

2. A particle as recited in claim 1; additionally comprising a plurality of drug molecules having general or specific toxicity against malignant mammalian tumors or metastases; wherein said drug molecules are directly bonded covalently to said iron oxide nanoparticle, or are directly bonded to said ligand molecules, or both; and wherein the plurality of drug molecules may be the same or different.

3. A particle as recited in claim 1, wherein said iron oxide nanoparticle comprises Fe₃O₄.

4. A particle as recited in claim 1, wherein said iron oxide nanoparticle comprises Fe₂O₃ or FeO.

5. A particle as recited in claim 1, wherein the diameter of said particle is between about 1 nm and about 400 nm.

6. A particle as recited in claim 1, wherein the diameter of said particle is between about 5 nm and about 150 nm.

7. A particle as recited in claim 1, wherein the diameter of said particle is between about 10 nm and about 100 nm.

8. A particle as recited in claim 1, wherein the diameter of said particle is about 10 nm.

9. A composition comprising a plurality of particles as recited in claim 1.

10. A particle as recited in claim 1, wherein said particle consists essentially of said iron oxide nanoparticle, said ligand molecules, and linking groups covalently bonded to the surface of said iron oxide nanoparticle and to said ligand molecules; and wherein amino groups, hydroxyl groups, or other low-molecular weight unbound linking moieties may optionally be present that are bound to said iron oxide nanoparticle but that are not bound to one of said ligand molecules; and wherein said particle is essentially free from any groups that are covalently bound to said iron oxide nanoparticle other than said ligand molecules and said optional low-molecular weight unbound linking moieties.

11. A particle as recited in claim 2, wherein said particle consists essentially of said iron oxide nanoparticle, said ligand molecules, said drug molecules, and linking groups covalently bonded to the surface of said iron oxide nanoparticle and to said ligand molecules, said drug molecules, or both; and wherein amino groups, hydroxyl groups, or other low-molecular weight unbound linking moieties may optionally be present that are bound to said iron oxide nanoparticle but that are not bound to one of said ligand molecules or said drug molecules; and wherein said particle is essentially free from any groups that are covalently bound to said iron oxide nanoparticle other than said ligand molecules, said drug molecules and said optional low-molecular weight unbound linking moieties.

12. A composition comprising a plurality of particles as recited in claim 2.

13. A composition as recited in claim 1, wherein said ligand molecules are covalently bonded to a spacer molecule, and said spacer molecule is covalently bonded to said iron oxide nanoparticle.

14. A method for in vivo imaging in a mammal of cells or tissues that express a selected receptor; said method comprising the steps of:
(a) administering to the mammal a composition as recited in claim 10, wherein the ligand molecules are specific for the selected receptor;
(b) waiting a time sufficient to allow the ligands to bind to the selected receptors; and
(c) imaging the cells or tissues with a non-invasive imaging technique whose resolution is enhanced by the presence of the iron oxide on or within the cells.

15. A method as recited in claim 14, wherein the imaging technique is selected from the group consisting of magnetic resonance imaging, magnetic spectroscopy, X-ray, positron emission tomography, computer tomography, and ultrasonic imaging.

16. A method as recited in claim 14, wherein the imaging technique comprises magnetic resonance imaging.

17. A method as recited in claim 14, wherein the selected receptor is specifically expressed by malignant cells, and wherein one or more tumors or metastases are imaged.

18. A method as recited in claim 14, wherein the selected receptor is specifically expressed by malignant cells, and wherein one or more individual malignant cells or nonvascularized malignant cell clusters are imaged.

19. A method as recited in claim 14, wherein the ligand molecules comprise luteinizing hormone releasing hormone; and wherein one or more tumors, metastases, nonvascularized malignant cell clusters, or individual malignant cells are imaged, selected from the group consisting of breast cancer, ovarian cancer, prostate cancer, lung cancer, pancreatic cancer, endometrial cancer, colon cancer, non-Hodgkin's lymphoma, brain cancer, oral cancer, hepatic cancer, and renal cancer.

20. A method as recited in claim 14, wherein the ligand molecules comprise Her2/neu; and wherein one or more breast or prostate tumors, metastases, nonvascularized malignant cell clusters, or individual malignant cells are imaged.

21. A method as recited in claim 14, wherein the ligand molecules comprise transferrin; and wherein one or more colon or bladder tumors, metastases, nonvascularized malignant cell clusters, or individual malignant cells are imaged.

22. A method as recited in claim 14, wherein the ligand molecules comprise folate; and wherein one or more lung, kidney, or colon tumors, metastases, nonvascularized malignant cell clusters, or individual malignant cells are imaged.

23. A method as recited in claim 14, wherein the ligand molecules comprise melanocyte stimulating hormone; and wherein one or more melanomas, metastases, nonvascularized malignant cell clusters, or individual malignant cells are imaged.

24. A method as recited in claim 14, wherein the ligand molecules comprise one or more of the compounds estradiol, testosterone, follicle stimulating hormone, and progesterone; and wherein one or more gonadal cancers, metastases, nonvascularized malignant cell clusters, or individual malignant cells are imaged.

25. A method as recited in claim 14, wherein the ligand molecules comprise an antibody or an antibody fragment with specific affinity for a selected receptor.

26. A method as recited in claim 14, wherein the ligand molecules comprise αᵥβ₃; and wherein one or more diseased cardiovascular tissues are imaged.

27. A method as recited in claim 14, wherein the ligand molecules comprise vasoactive intestinal peptide; and wherein one or more inflamed tissues are imaged.

28. A method as recited in claim 14, wherein the ligand molecules comprise a mixture of different ligand molecules.

29. A method as recited in claim 14, wherein the receptor mediates endocytosis; and wherein step (b) comprises waiting a time sufficient to cause the particles to be endocytosed by cells expressing the selected receptor.

30. A method for killing or inhibiting the growth of one or more tumors, metastases, nonvascularized malignant cell clusters, or individual malignant cells in a mammal; said method comprising administering to the mammal an effective amount of a composition as recited in claim 12, wherein the ligand molecules are specific for a receptor that is specifically expressed by the one or more tumors, metastases, or both.

31. A method as recited in claim 30, additionally comprising the step of imaging the cells or tissues with a non-invasive imaging technique whose resolution is enhanced by the presence of the iron oxide on or within the cells

32. A method as recited in claim 31, wherein the imaging technique is selected from the group consisting of magnetic resonance imaging, magnetic spectroscopy, X-ray, positron emission tomography, computer tomography, and ultrasonic imaging.

33. A method as recited in claim 32, wherein the imaging technique comprises magnetic resonance imaging.

34. A method as recited in claim 30, wherein the ligand molecules comprise luteinizing hormone releasing hormone; and wherein one or more tumors, metastases, nonvascularized malignant cell clusters, or individual malignant cells selected from the group consisting of breast cancer, ovarian cancer, prostate cancer, lung cancer, and pancreatic cancer are killed or inhibited.

35. A method as recited in claim 30, wherein the ligand molecules comprise Her2/neu; and wherein one or more breast or prostate tumors, metastases, nonvascularized malignant cell clusters, or individual malignant cells are killed or inhibited.

36. A method as recited in claim 30, wherein the ligand molecules comprise transferrin; and wherein one or more colon or bladder tumors, metastases, nonvascularized malignant cell clusters, or individual malignant cells are killed or inhibited.

37. A method as recited in claim 30, wherein the ligand molecules comprise folate; and wherein one or more lung, kidney, or colon tumors, metastases, nonvascularized malignant cell clusters, or individual malignant cells are killed or inhibited.

38. A method as recited in claim 30, wherein the ligand molecules comprise melanocyte stimulating hormone; and wherein one or more melanomas, metastases, nonvascularized malignant cell clusters, or individual malignant cells are killed or inhibited.

39. A method as recited in claim 30, wherein the ligand molecules comprise one or more of the compounds estradiol, testosterone, follicle stimulating hormone, and progesterone; and wherein one or more gonadal cancers, metastases, nonvascularized malignant cell clusters, or individual malignant cells are killed or inhibited.

40. A method as recited in claim 30, wherein the ligand molecules comprise an antibody or an antibody fragment with specific affinity for the receptor.

41. A method as recited in claim 30, wherein the ligand molecules comprise a mixture of different ligand molecules.

42. A method as recited in claim 30, wherein the receptor mediates endocytosis.
